SEQUENCE LISTING (16 sequences)

SEQ ID NO: 1 - RNA, 22 nt, Homo sapiens
ugagguaguagguugugugguu

SEQ ID NO: 2 - RNA, 22 nt, Homo sapiens
cuauacaaccuacugccuuccc

SEQ ID NO: 3 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 4 - DNA, 3913 bp, Homo sapiens
agcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaaaaaaaaaaa

SEQ ID NO: 5 - protein, 508 aa, Homo sapiens
MGSCCSCPDKDTVPDNHRNKFKVINVDDDGNELGSGIMELTDTELILYTRKRDSVKWHYLCLRRYGYDSNLFSFESGRRCQTGQGIFAFKCARAEELFNMLQEIMQNNSINVVEEPVVERNNHQTELEVPRTPRTPTTPGFAAQNLPNGYPRYPSFGDASSHPSSRHPSVGSARLPSVGEESTHPLLVAEEQVHTYVNTTGVQEERKNRTSVHVPLEARVSNAESSTPKEEPSSIEDRDPQILLEPEGVKFVLGPTPVQKQLMEKEKLEQLGRDQVSGSGANNTEWDTGYDSDERRDAPSVNKLVYENINGLSIPSASGVRRGRLTSTSTSDTQNINNSAQRRTALLNYENLPSLPPVWEARKLSRDEDDNLGPKTPSLNGYHNNLDPMHNYVNTENVTVPASAHKIEYSRRRDCTPTVFNFDIRRPSLEHRQLNYIQVDLEGGSDSDNPQTPKTPTTPLPQTPTRRTELYAVIDIERTAAMSNLQKALPRDDGTSRKTRHNSTDLPM

SEQ ID NO: 6 - DNA, 6676 bp, Homo sapiens
aaaacccttccctcccccgctcccccggaagtgcttttccaagattcgggccggagagaggccttgtaggcacagcggctgagactcgatctgctccaagtaggggctccagcgcgggtcggagtctgggggttcgcgcccgccgacccgcgccctgctccctctcagcacctgggcggacggttaaatcagcaaacaaagaaaacatggtattttgaaatatgattaaactcctgatgctgcagcagaggctaagaatattaatggccagatctagtgcacacatggtcttctgaagaagccatgggtagctgttgtagctgtccagataaagacactgtcccagataaccatcggaacaagtttaaggtcattaatgtggatgatgatgggaatgagttaggttctggcataatggaacttacagacacagaactgattttatacacccgcaaacgtgactcagtaaaatggcactacctctgcctgcgacgctatggctatgactcgaatctcttttcttttgaaagtggtcgaaggtgtcaaactggacaaggaatctttgcctttaagtgtgcccgtgcagaagaattatttaacatgttgcaagagattatgcaaaataatagtataaatgtggtggaagagccagttgtagaaagaaataatcatcagacagaattggaagtccctagaacacctcgaacacctacaactccaggatttgctgctcagaacttacctaatggatatccccgatatccctcatttggagatgcttcatcccatccgtcaagcagacatccttctgtgggaagtgctcgcctgccttcagtaggggaagaatctacacatcctttgcttgtggctgaggaacaagtacatacctatgtcaacactacaggtgtgcaagaagagcggaaaaaccgcacaagtgtgcatgttccattggaggcgagggtttctaacgctgaaagcagcacaccaaaagaagaaccaagtagtattgaggacagggatcctcagattcttcttgaacctgaaggagtcaaatttgttttagggccaacccctgttcaaaagcagttaatggaaaaagagaaactggagcaacttggaagagatcaagttagtggaagtggagcaaataacacagaatgggacactggctatgacagtgatgaacgaagagatgcaccctctgttaacaaactggtgtatgaaaatataaatgggctatctatccctagtgcctcaggggtcaggagaggtcgtctgacatccaccagtacctcagatacccagaatatcaacaactcagctcagagaagaactgcattattaaactatgaaaatctaccatctttgcctcctgtttgggaagcccgcaagctaagtagggatgaagatgacaatttaggaccaaagaccccatctctaaatggctaccataataatctagatccaatgcataactatgtaaatacagagaatgtaacagtgccagcaagtgctcacaaaatagaatattcaaggcgtcgggactgtacaccaacagtctttaactttgatatcagacgcccaagtttagaacacaggcagcttaattacatacaggttgacttggaaggtggcagtgactctgacaaccctcagactccaaaaacgcctacaactccccttccacaaacccctaccaggcgcacagagctgtatgccgtgatagacatcgagagaactgctgctatgtcaaatttgcagaaagcactgccacgagatgatggtacatctaggaaaactagacacaatagtactgatctgcccatgtgagcctggaaagcattgtgttgtttgcacctttgtgaagtttttaaaaatgaagatgcaagtgcttcattttcatttctaaacactaactccttttatagactgataaaatttttttctgaatatttcatgtgcatctttaactaaagggaattaatgtagagcaggtactccttaaagaacactaatttcattatatactactcgttgtacagcagcattcccgttttcacagtgcctatttaaaatgagagttgaagtaaatgacatgctggttgatttttatcaatattctggacttaacgcatacctttcatgtctaagtcatggttggcttttaaaactttttataaagcctcttgacaatgtacattgctaacaggtaactataggctttgaaagtaatgctcgtagattcagtgttcacagtatgtggcctccagcatgtaacatgaggaatcctttatttcattaattaatggctttttgacttgagccaaaacatatgtaaaggaaacagaagtaccgcacctcctcttacaccagtcagctcctttgccttcagtgttactagaaagcggcctgtgtccatgagtgtgctttgctgttggtgcactgaaaggcaggaaggagacaagattttctatttactcatctcatgatgtcatttgaagggcatgtccagatatcttaaaattataataggctcaagaatcagtctcaggtcactttacccaaaaacatttgaaaatctgaaccacaatctcctgaaagtttttctcctatagattgttgacaacacattgttttctggaggcatttgtgccattaggtttccatttatcttcagtttttttctttggtgtttgggatgtcttattttgttgccttatgtccttttcaatttaaaatgtttgagtttgtatatagttttgaaattggattatgtgttcattgttgtttagtttgcatttttgtcaaattatggttttgaaggttcatttggaacttactgttagtctgtaacagggttgcccttgtccagtatttatttataagctgtttacttttcaagttgataaaaacattctccaattctaaatttgcttgtgtccataggtgatctctttagcaaactgagaaaaaaaggaagctacttttaacatgcaaagttccctcaaggtgtaccgtgttgtctctgtgggcactcttccccagcactttagcagtaattcccccagctacacgctgcagttgtactctgcccactctagtgttcctcagctctgctgtccttttacttgtagctggatctttgattatccttcgatttccatgaaatattaatattgttgccagcatagcaggtacagtggaagtcttgtagcagtgagattgtatcataatttaggatttaaaatgaattaaagtttatataaactgaagagtctccatatgtcaaactcttggaaaatcaaagatgttccaatttcctaaacactagagaatacgagagaaggtagagtggaaaaggttaggtaaccttgcaaaatattttactattttctctaaatatgaggaagtttgagattatgatctggatctaccagatataactaaggttaatttagcatgaaaaagttttagtcatattggcatccaacctattcagtaaccgaatcataggacaatgatggattaggagaacaatagagtgggatcattataaagaaaataaattattaaaggtgtctttatcgttttagtgccatttttagtgtctttactataaatcaatatcagtgtattttatcattctatgtgcatagcagaattttcttttctcccttttgttcccctgtgaacttggtgcttattaaagtgctcactgttctcttaaaagagagcagtggtataggtgtgcagtttccatgatgcaggttccatttttaatatattgttccacttatcctttcttctgagtaaattgctaattgtgccaaatttatgtaatagtttttgtaatgtggaataagaattatgatggaaccattgcacatttttttctgaaacagccagtcaaggcagaacattaatctccaaatgcaagggctgatctatttattcattttggaggttgggtactttattctttctttccgtcatccttttcattgtttcccccggattctaattagtttttattttttttagataactccaatataatcattacagtttatgctttaaatactatgtgctttaaaaaggaaaatgggaccaatttgtctgctaagaatttgattttaggtactataagagtattaggaaaatatatacaactggtgttaatttctagatattttctagaaatcacttgtgttcctatttaataaaaggtaatttagaatactacttgtcctttgcagtagtttagtaatgggcattaagctgtgtcctcgaaggatgtacctattactaggtgcattttagaatgaaatattgatattttattagcatataattgtggccatatatctcagattttctgaggcagatctaattttagataattctgttggtagaccatgtgatccttctttttggttttggaaatataatcattgttaatgttttccctccaaatagaatactgttttatccatacaaatcataacagcatctatcccatgctagggttggaaactgatattggtattacttgtgttttttcttagtgtgttttatttcccagtttcatcttcttctaaaaatgaaaatatggtgccttccctccctccaggaagactggcaaatatttccttttatttactgctgctgtggagtgatgagatatgcactttactctttaagattcagcaaaaagcttttcacttctcagtatatccagaatacatcatatctgggacttaggaaaatttgccaagcaatctttgtttttatagatactaatgttgaccctctccagcgttcaatgttataaatagaacaagtcaagctagtgtttatctcctccccctccccaaaactgtggcacagcatataaaaatgtacctcaataatgttctattaaaaatgggacaggggccttatgttttcataatttcccaacaatgtgccgccatatttttgcctcaaggtaaaggttttaacagatgaaaaagtacttcccaattcccccgtgctattcctaacctataatgcccaaatgttttgtgcaatgtgtagtgtgtgtgtataaatacatatattcttgaaatagacataccatcagagacatcattcacaagtaactgatgtattggcatctcattcatatttctgatgtgtgaggtatatggtactaattaccttttccttgatgtttgccaaatttgaataaaggcattggtacgaaattacagaatgtaaagaaaatgtttttggcttgaaaaattaacatattttatgacgtaccacagtatactctgcccaaaccagcaccctatctatctttcctgttctttacatccctgttccccatccctacttcctcatttttggtataacacagttcttttgtagcatcattataattgcagttctatggcaattggacagttatagcatggaaacagactggtataagtagtacagtagtcaccagtgtgccacatttgcattagtaatgcaaaatatacattttataaaggacaaactttgtgttatgttttattttcattacattgtataatattgtaagactattgtatgtcctaatttgcattataaatgtttttttcctacgtaaaggcataaatatagcaactttgtataaaggtagcttattagatttttaattttttcttttataaaaaattgtccaacagtgggactaccattgccaaattgtatatgaaatatgaattttacccccatggttaatttcttttataaacattccatatttctctaataaaaagacataagtgatactgtactatgcatacattgtatcttaatgctgtttcagatcagcattttaaattttggtttgcatttttaatattggcaaaacgtaaccactgttaattaaaataaaaccttgttgtatatgtaacaacataattttccctctatcccttcccaccctttgttctctatttctccctatcagtgccaacttcatacattttgtagcatggcaataaaatataacttttacactgaggccgagtgtggctttttggaggaagtggggatgggacgattgccctctagttgtcctttgcatatgactgttttttgccatataagccatgtcatcaggcatgaaaagttttctcatatatgatgtaaacttgcttttaaggacaagtgtgaatgtgctttttaagcttaatttttgtcatgacaactaattttttttatctttggagaagtcagagttctttacaatcaaacgtttattaactggagtacttagaataagctagtaattgaatttagttcaagggctaagcaacacatttttaaatccttatttattgtagagtattagtatactgtcctacaaattatgtaaaatatggtttaatattagatgactttggattttgcaatgccttactgttgtcattctagcataaatatccataatgaggtactcaagttgatactggaagctgagctgatcatacactgacctgaagcattcatgaaaagctgctttattgaataaagtctgattggagttcttttcatgctcactttccccttattgctgaaagtagattgcaataaaaccccaataaaacgtttggtcggatatctacttaaaaaaaaaaaa

SEQ ID NO: 7 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 8 - DNA, 1746 bp, Homo sapiens
cccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggaaaaaaaaaaaaaaaaaaaaaaaaaaaaaaaaaaaaaaaaaaaaaaaaaaaaaaaaaaaaaaaaaaa

SEQ ID NO: 9 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 10 - DNA, 2570 bp, Homo sapiens
gcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcag

SEQ ID NO: 11 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 12 - DNA, 930 bp, Homo sapiens
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtag

SEQ ID NO: 13 - protein, 426 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 14 - DNA, 5766 bp, Homo sapiens
gcggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatttctgccttttaagatatgtacagaaaatgtccatataaatttccattgaagtcgaatgatactgagaagcctgtaaagaggagaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaaaaaaaaaaaaaaaaaaaaaaaaaaaaaaagaaaaaaaaaaaaaaa

SEQ ID NO: 15 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 16 - DNA, 487 bp, Homo sapiens
cctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgggtgagtggtccccagcccgggctcggcggggcgccgggggtcttcctggggtccccgcctctccgctgcgcttgacagtcgggcccggcaacccggcccccgggcggaaacgaggaaagtttcccccgcgacactcacgcagcccgactcccgtagctgcagggattgtgagtttttcttgaaaaagagaaggaaagttcagttgcaaggggcgcggggcacgtttggtcc

(19) United States
(12) Patent Application Publication
(10) Pub. No.: US 2022/0233573 A1
(43) Pub. Date: Jul. 28, 2022

(54) METHODS AND COMPOSITIONS FOR TREATING PULMONARY ARTERIAL HYPTERNSION

(71) Applicant: Yale University, New Haven, CT (US)

(72) Inventor: Michael SIMONS, New Haven, CT (US)

(21) Appl. No.: 17/626,581

(22) PCT Filed: Jul. 14, 2020
(86) PCT No.: PCT/US2020/041957

§ 371 Date: Jan. 12, 2022

Related U.S. Application Data
(60) Provisional application No. 62/874,322, filed on Jul. 15, 2019.

(51) Int. Cl. A61K 31/713; A61K 31/436; A61K 45/06; A61P 9/00; A61P 9/02; C12N 15/113

(52) U.S. Cl. CPC A61K 31/713; A61K 31/436; A61K 45/06; C12N 2310/321; A61P 9/02; C12N 15/113; C12N 2310/141; A61P 9/00

(57) ABSTRACT

In various aspects and embodiments, the invention provides methods of treating pulmonary arterial hypertension by inhibiting the endothelial to mesenchymal transition. The invention provides a method of treating pulmonary arterial hypertension (PAH) in a subject, the method comprising administering to the subject an agent that modulates the activity or level of let-7 mlRNA in an endothelial cell in the subject, thereby treating PAH in the subject. In another aspect, the invention provide a method of treating PAH in a subject, the method comprising administering to the subject an agent that decreases the activity or level of an endothelial TGFβ signaling polypeptide or a TGFβ peptide receptor, thereby treating PAH in the subject.

### CROSS-REFERENCE TO RELATED APPLICATIONS

The present application claims priority under 35 U.S.C. § 119(e) to U.S. Provisional Patent Application No. 62/874,322 filed Jul. 15, 2019, which is incorporated herein by reference in their entirety.

### STATEMENT REGARDING FEDERALLY SPONSORED RESEARCH OR DEVELOPMENT

This invention was made with government support under HL135582 awarded by National Institutes of Health. The government has certain rights in the invention.

### BACKGROUND OF THE INVENTION

Pulmonary arterial hypertension (PAH) is a significant health problem. Current methods of treatment and prevention are inadequate. There is a need in the art for methods of treating PAH. This disclosure addresses that need.

### SUMMARY OF THE INVENTION

In one aspect, the invention provides a method of treating pulmonary arterial hypertension (PAH) in a subject, the method comprising administering to the subject an agent that modulates the activity or level of let-7 miRNA in an endothelial cell in the subject, thereby treating PAH in the subject.

In another aspect, the invention provide a method of treating pulmonary arterial hypertension (PAH) in a subject, the method comprising administering to the subject an agent that decreases, in an endothelial cell in the subject, the activity or level of a endothelial TGFβ signaling polypeptide or TGFβ peptide receptor selected from the group consisting of TGFβ1, TGFβ2, TGF033, TGFβR1, and TGFβR2, thereby treating PAH in the subject.

In yet another aspect, the invention provides a method of treating pulmonary arterial hypertension (PAH) in a subject, the method comprising administering to the subject an agent that decreases, in an endothelial cell in the subject, the activity or level of FRS2α, thereby treating PAH in the subject.

In certain embodiments, the agent is selectively delivered to an endothelial cell in the subject. In certain embodiments, the agent is in a nanoparticle. In certain embodiments, the nanoparticle is a 7C1 nanoparticle.

In certain embodiments, the agent is selectively delivered to a smooth muscle cell in the subject.

In certain embodiments, the agent is administered intravenously.

In certain embodiments, the agent that increases the activity or level of let-7 miRNA is selected from the group consisting of human let-7b miRNA and human let-7c miRNA.

In certain embodiments, the agent that modulates the activity or level of let-7 miRNA is a pharmaceutical composition comprising an effective amount of a let-7 miRNA in a nanoparticle formulated for selective delivery to an endothelial cell, in a pharmaceutically acceptable excipient.

In certain embodiments, the let-7 miRNA comprises a chemical modification that increases stability of the miRNA and/or reduces an immune response to the miRNA in a subject. In certain embodiments, the chemical modification is a 2′-O-methyl modification.

In certain embodiments, the let-7 miRNA is selected from the group consisting of human let-7b miRNA and human let-7c miRNA.

In certain embodiments, the agent that decreases the activity or level of a TGFβ signaling polypeptide is an inhibitory polynucleotide that reduces expression of the TGFβ signaling polypeptide.

In certain embodiments, the agent that decreases the activity or level of FRS2α is an inhibitory polynucleotide that reduces expression of a FRS2α polypeptide.

In certain embodiments, the decrease in the activity or level of the FRS2α polypeptide promotes smooth muscle cell proliferation.

In certain embodiments, the method further comprising providing to the subject a second therapeutic agent comprising an mTOR inhibitor. In certain embodiments, the mTOR inhibitor is rapamycin.

### BRIEF DESCRIPTION OF THE DRAWINGS

FIGS. 1A-1B show a time course of development of spontaneous PAH in mice after endothelial-specific deletion of MEKK3. FIG. 1A: Representative right ventricular systolic pressure (RVSP) tracing in control and MEKK3 iEC−/− mice 6 weeks after MEKK3 deletion. FIG. 1B: Time course of RVSP increase.

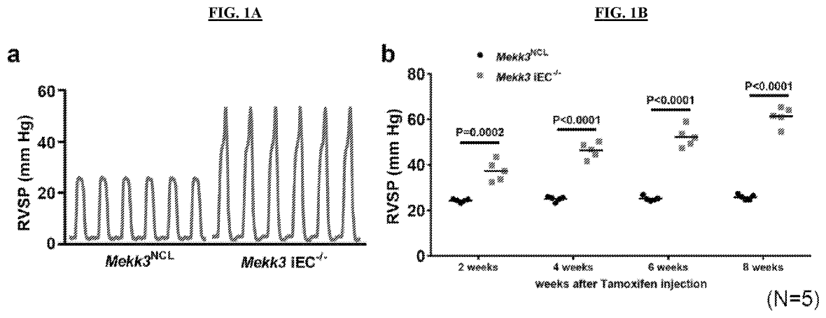

FIG. 2 depicts a right ventricular (RV) hypertrophy in MEKK3 ECKO mice. RV-right ventricular thickness; LV: left ventricular free wall thickness. S-interventricular septum thickness

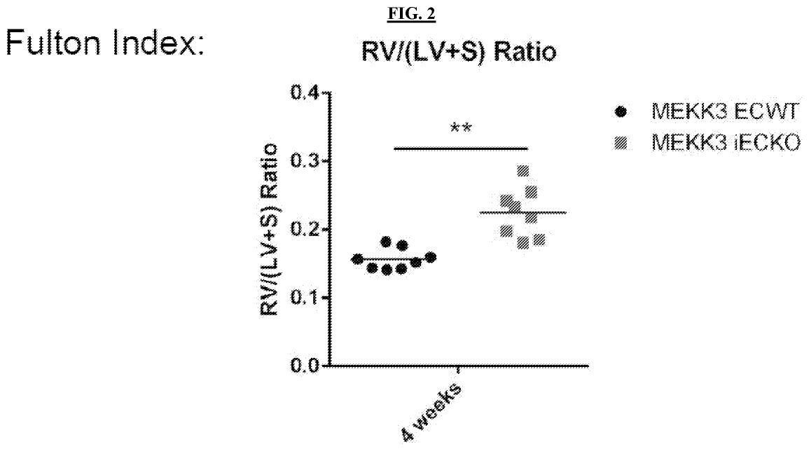

FIGS. 3A and 3B show morphologic evidence of PAH in MEKK3 ECKO mice. FIG. 3A: total lung fields section stained with anti-SMA antibody. Note increased SMA staining in peripheral lung fields indicating hypertrophy of small pulmonary arteries; FIG. 3B: vibratome section.

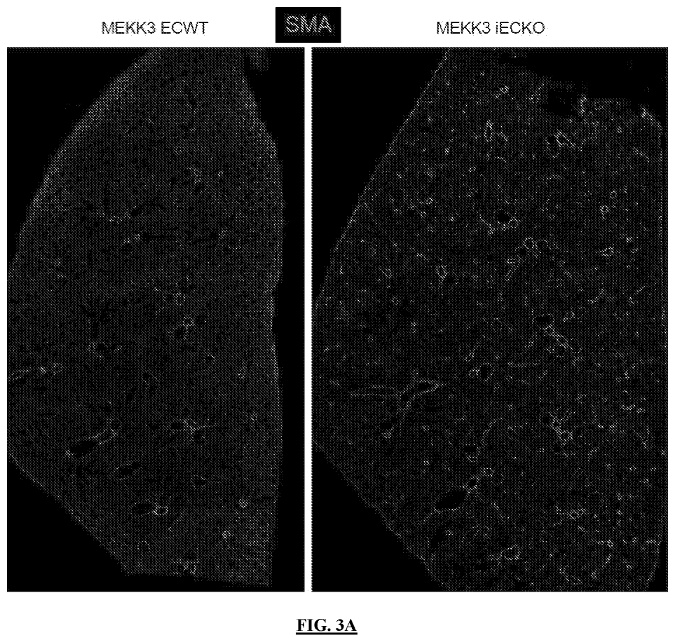

FIGS. 4A and 4B depict fate-mapping of cells giving origin to PAH in MEKK3 ECKO mice. Mice carrying Cdh5-Cre (endothelial-specific Cre) were crossed with mTmG reporter mice and MEKK3 fl/fl mice. This fate-maps all endothelial cells as green. Following MEKK3 deletion these former EC are expressing smooth muscle markers showing that EC-to-SMC fate change drives PAH.

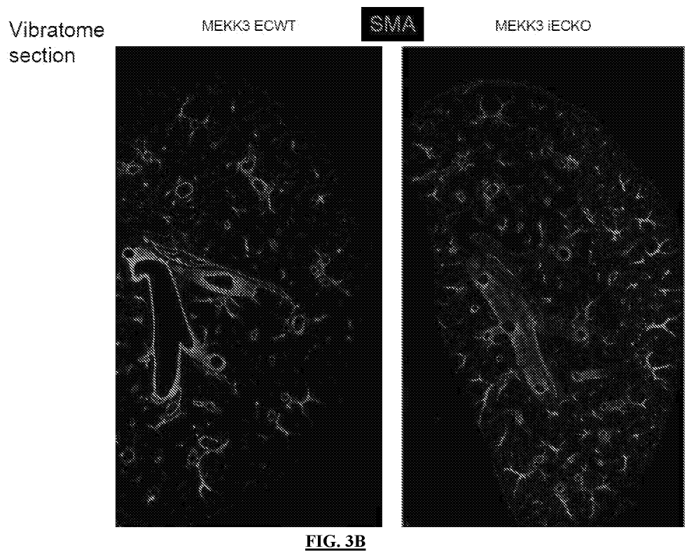

FIG. 5 depicts RNA sequencing of human umbilical vein endothelial cells (HUVEC) after MEKK3 knockdown relative to control.

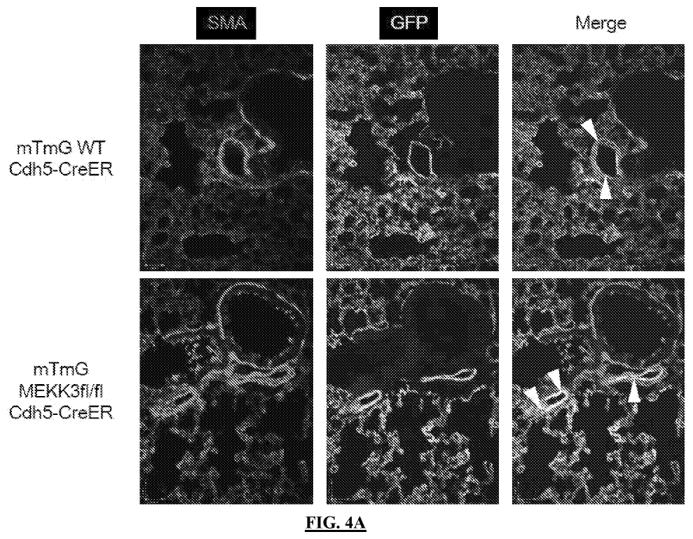

FIGS. 6A-6B show that MEKK3 knockdown induces EndMT in vitro. FIG. 6A: RNA-seq analysis of gene expression showing increased EndMT; FIG. 6B: Western blot analysis.

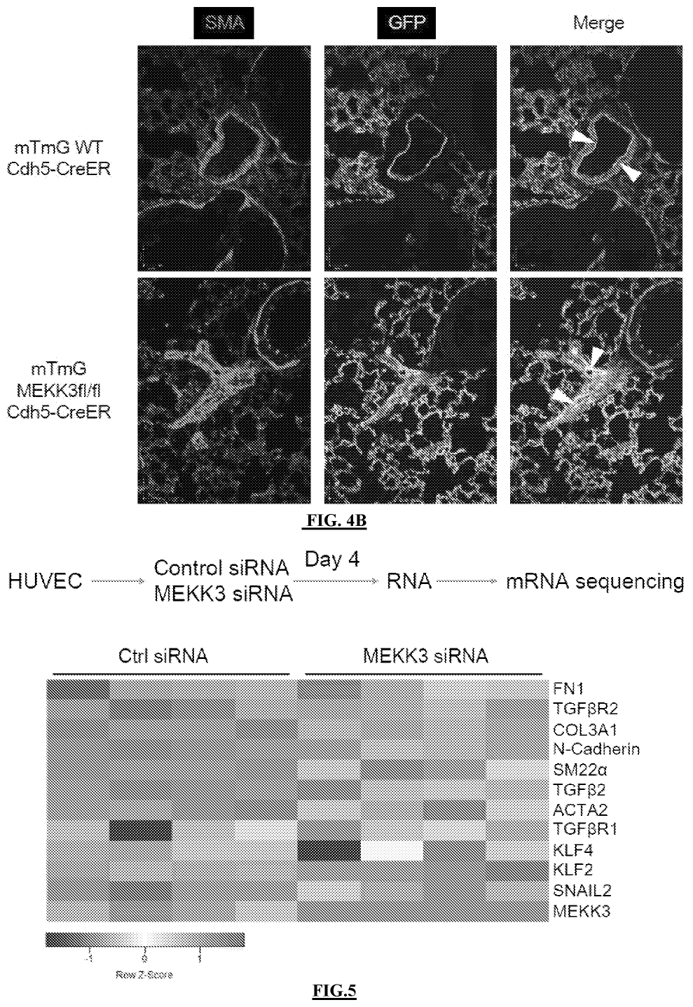

FIG. 7 depicts increased EndMT after MEKK3 knockdown.

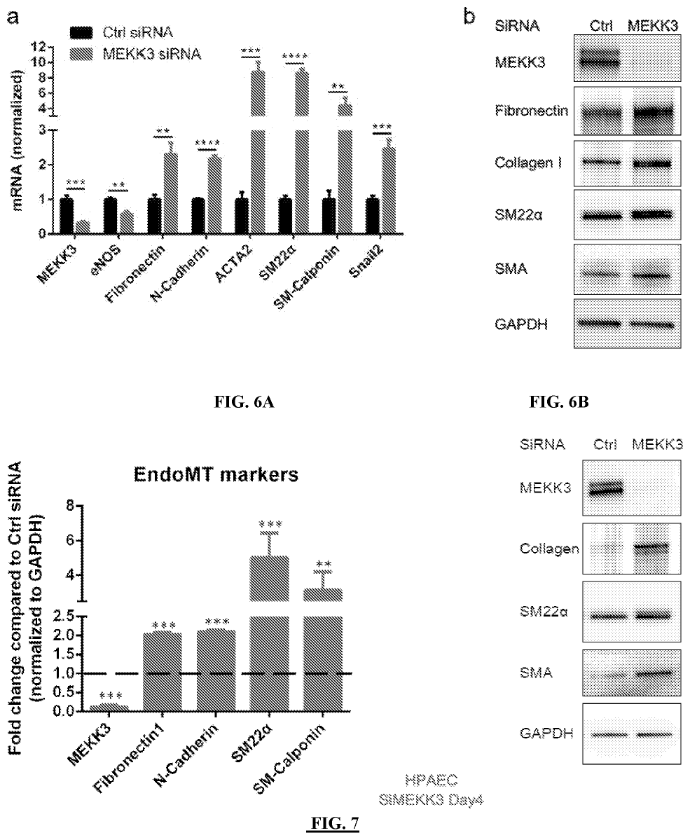

FIG. 8 depicts increased EndMT in vivo in MEKK3 ECKO mice: note increased TGFbR2 expression in pulmonary ECs.

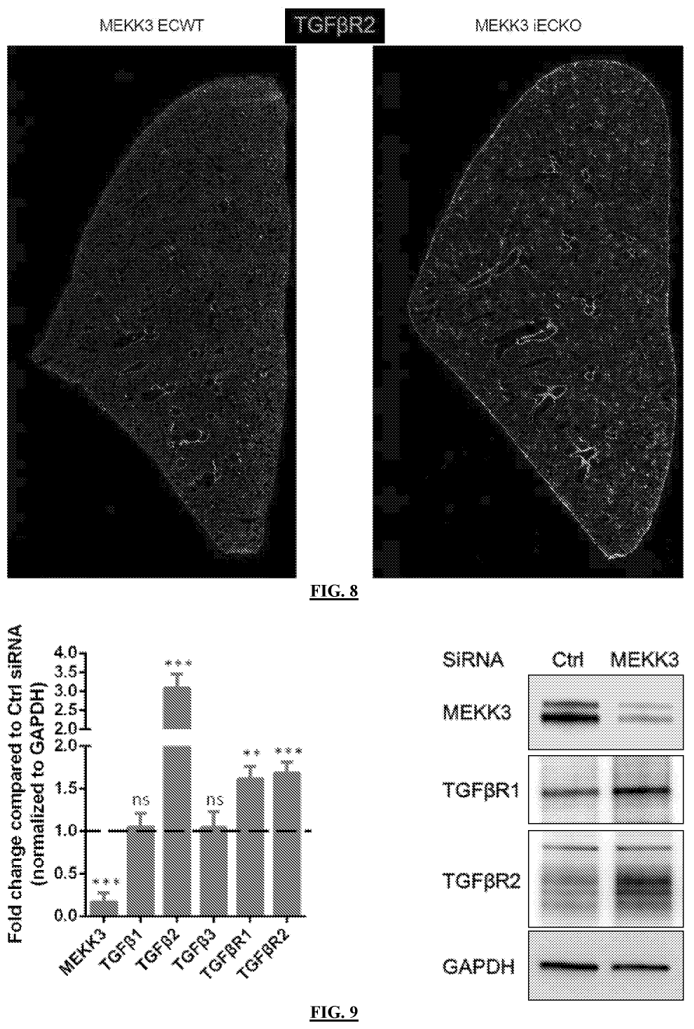

FIG. 9 depicts increased TGFb and TGFbR genes expression after MEKK3 KD in ECs.

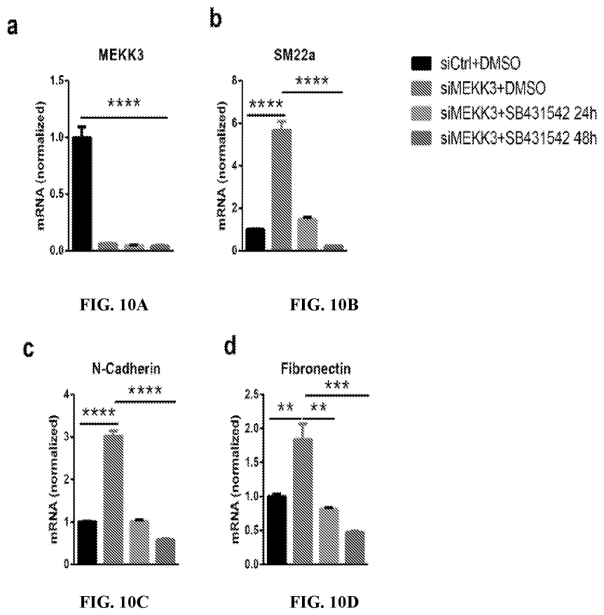

FIGS. 10A-10D depict EndMT after MEKK3 KD.

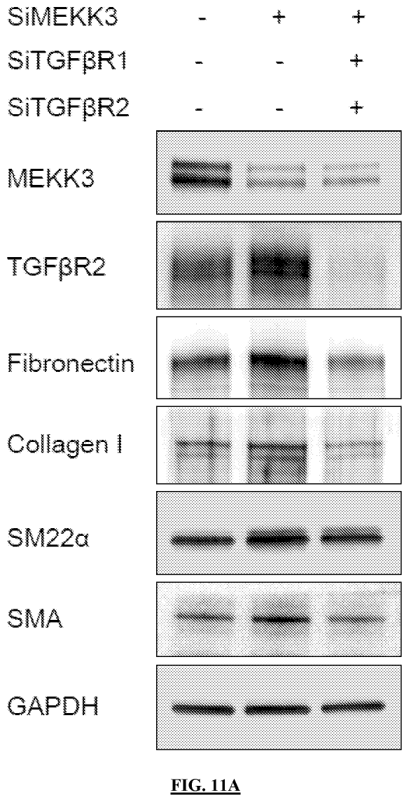

FIGS. 11A-11B TGFbR1/R2 knockdown suppresses MEKK3 KD-induced EndMT.

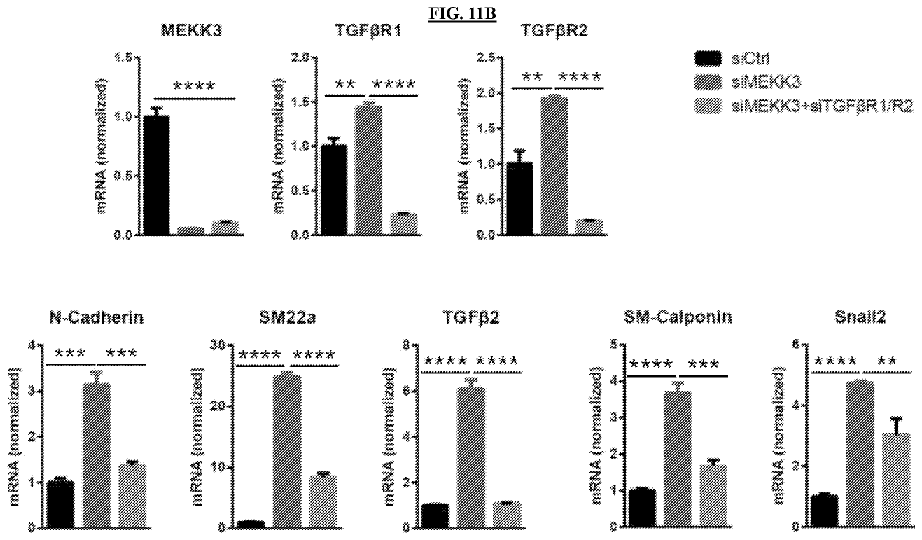

FIGS. 12A-12D depict nanoparticle (7C1)-delivered siRNA to TGFbR1 and TGFbR2 prevents development of PAH. FIG. 12A: Time course and experiment design. FIG. 12B: RVSP tracings 3 weeks after MEKK3 KO induction along with NP-based TGFbR1/R2 treatment. FIG. 12C: Quantification of RVSP. FIG. 12D: Reduction in RV hypertrophy

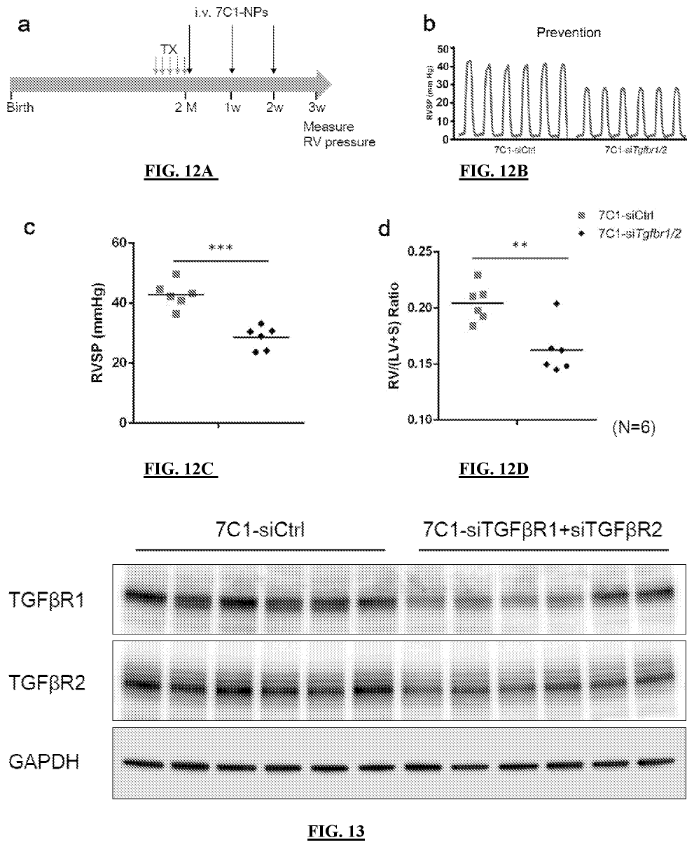

FIG. 13: Reduced EndMT in the pulmonary vasculature of MEKK3 ECKO mice after TGFbR1/R2 RNAi treatment

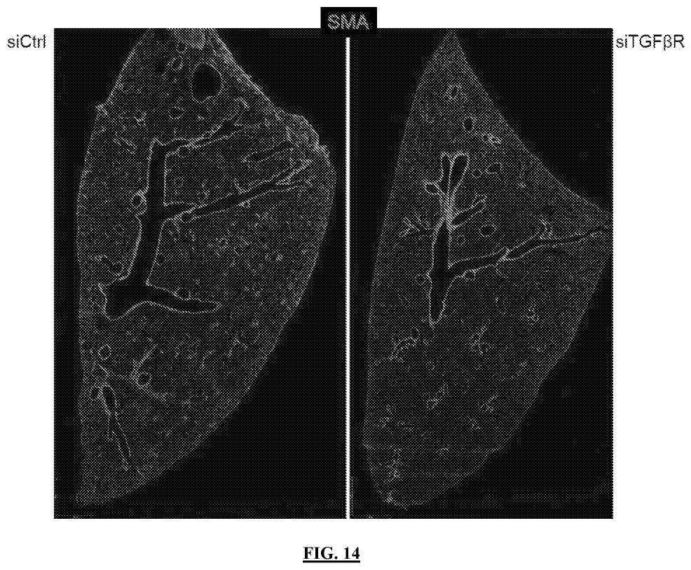

FIG. 14 is an image showing lungs treated with siTGFβR and control, stained for SMA.

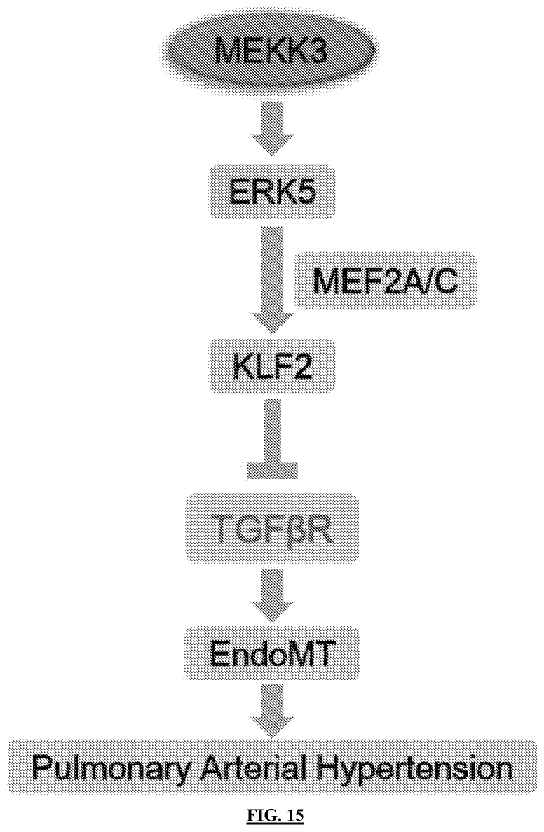

FIG. 15 depicts proposed mechanism of action FIG. 16: MEKK3 KO reduces endothelial let-7 levels.

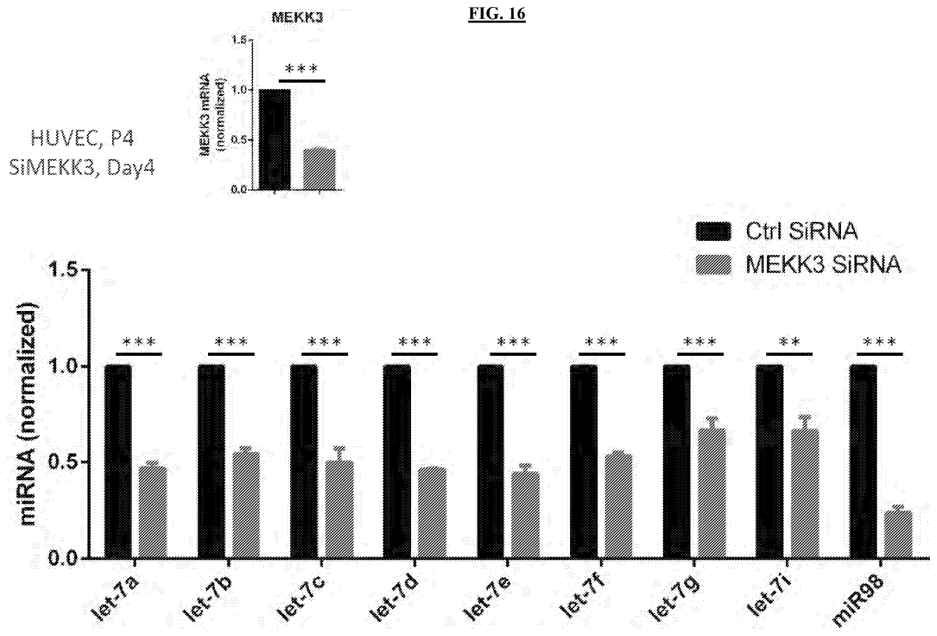

FIGS. 17A and 17B depict EC-specific TGFbR2 KO prevents PAH development in MEKK3 ECKO mice.

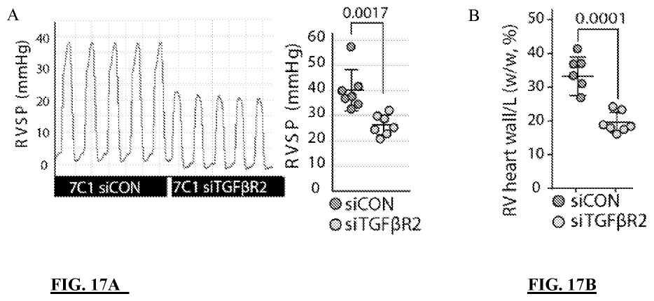

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

By “agent” is meant any small molecule chemical compound, antibody, nucleic acid molecule, or polypeptide, or fragments thereof. In some embodiments, the agent is a nucleic acid molecule.

By “alteration” is meant a change (increase or decrease) in the expression levels or activity of a gene or polypeptide as detected by standard art known methods such as those described herein. In some embodiments, an alteration in expression level includes a 10% change in expression levels, a 25% change, a 40% change, and a 50% or greater change in expression levels.

“Biological sample” as used herein means a biological material isolated from a subject, including any tissue, cell, fluid, or other material obtained or derived from the subject. In some embodiments, the subject is human. The biological sample may contain any biological material suitable for detecting the desired analytes, and may comprise cellular and/or non-cellular material obtained from the subject. In certain embodiments, the biological sample is an endothelial cell. Biological samples include tissue samples (e.g., cell samples, biopsy samples), such as tissue from the heart or aorta. Biological samples also include bodily fluids, including, but not limited to, blood, blood serum, plasma, saliva, and urine.

By “capture reagent” is meant a reagent that specifically binds a nucleic acid molecule or polypeptide to select or isolate the nucleic acid molecule or polypeptide. In some embodiments, the capture reagent is a probe or primer that specifically binds a polynucleotide encoding a TGFβ signaling polypeptide, a let-7 miRNA, or a FGF signaling polypeptide.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected. In some embodiments, a level of a let-7 miRNA, a TGFβ signaling polypeptide or polynucleotide, or a FGF signaling polypeptide or polynucleotide is detected.

By “disease” is meant any condition or disorder that damages or interferes with the normal function of a cell, tissue, or organ. Examples of diseases include atherosclerosis, pulmonary hypertension, and chronic inflammation induced fibrosis.

By “effective amount” is meant the amount of a required to ameliorate the symptoms of a disease relative to an untreated patient. In particular embodiments, the disease is PAH. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, a “FGF signaling polypeptide” is meant a member or component of a fibroblast growth factor (FGF) signaling pathway. In some embodiments, the FGF signaling polypeptide is FGFR1 polypeptide or FRS2α polypeptide.

By “FGFR1 polypeptide” is meant a polypeptide or fragment thereof having at least about 85% amino acid identity to GenBank Accession No. AAH15035.1 and having a biological activity of a FGFR1 polypeptide. Biological activities of a FGFR1 polypeptide include cell surface receptor activity and tyrosine kinase activity. The sequence at GenBank Accession No. AAH15035.1 is shown below (SEQ ID No: 3):

| | |
|---|---|
| 1 | mwswkcllfw avlvtatlct arpsptlpeq aqpwgapvev |
| | esflvhpgdl lqlrcrlrdd |
| 61 | vqsinwlrdg vglaesnrtr itgeevevqd svpadsglya |
| | cvtsspsgsd ttyfsvnvsd |
| 121 | alpssedddd dddssseeke tdntkpnrmp vapywtspek |
| | mekklhavpa aktvkfkcps |
| 181 | sgtpnptlrw lkngkefkpd hriggykvry atwsiimdsv |
| | vpsdkgnytc iveneygsin |
| 241 | htyqldvver sphrpilqag 1panktvalg snvefmckvy |
| | sdpqphiqwl khievngski |
| 301 | gpdnlpyvqi lktagvnttd kemevlhlrn vsfedageyt |
| | clagnsigls hhsawltvle |
| 361 | aleerpavmt splyleiiiy ctgafliscm vgsvivykmk |
| | sgtkksdfhs qmavhklaks |
| 421 | iplrrqvsad ssasmnsgvl lvrpsrlsss gtpmlagvse |
| | yelpedprwe 1prdrlvlgk |
| 481 | plgegcfgqv vlaeaigldk dkpnrvtkva vkmlksdate |
| | kdlsdlisem emmkmigkhk |
| 541 | niinllgact qdgplyvive yaskgnlrey lqarrppgle |
| | ycynpshnpe eqlsskdlvs |
| 601 | cayqvargme ylaskkcihr dlaarnvlvt ednvmkiadf |
| | glardihhid yykkttngrl |
| 661 | pvkwmapeal fdriythqsd vwsfgvllwe iftlggspyp |
| | gvpveelfkl lkeghrmdkp |
| 721 | snctnelymm mrdcwhavps qrptfkqlve dldrivalts |
| | nqeyldlsmp ldqyspsfpd |
| 781 | trsstcssge dsvfsheplp eepclprhpa qlangglkrr |

By “FGFR1 polynucleotide” is meant a polynucleotide encoding a FGFR1 polypeptide. An exemplary FGFR1 polynucleotide sequence is provided at GenBank Accession No. BC015035.1. The exemplary sequence provided at GenBank Accession No. BC015035.1 is reproduced below (SEQ ID No: 4).

| | |
|---|---|
| 1 | agcgctcttg cggccacagg cgcggcgtcc tcggcggcgg |
| | gcggcagcta gcgggagccg |
| 61 | ggacgccggt gcagccgcag cgcgcggagg aacccgggtg |
| | tgccgggagc tgggcggcca |
| 121 | cgtccggacg ggaccgagac ccctcgtagc gcattgcggc |
| | gacctcgcct tccccggccg |
| 181 | cgagcgcgcc gctgcttgaa aagccgcgga acccaaggac |
| | ttttctccgg tccgagctcg |
| 241 | gggcgccccg cagggcgcac ggtacccgtg ctgcagtcgg |
| | gcacgccgcg gcgccggggc |
| 301 | ctccgcaggg cgatggagcc cggtctgcaa ggaaagtgag |
| | gcgccgccgc tgcgttctgg |
| 361 | aggagggggg caccagctcc ggctccattg ttcccgcccg |
| | ggctggaggc gccgagcacc |
| 421 | gagcgccgcc gggagtcgag cgccggccgc ggagctcttg |
| | cgaccccgcc aggacccgaa |
| 481 | cagagcccgg gggcggcggg ccggagccgg ggacgcgggc |
| | acacgcccgc tcgcacaagc |
| 541 | cacggcggac tctcccgagg cggaacctcc acgccgagcg |
| | agggtcagtt tgaaaaggag |
| 601 | gatcgagctc actgtggagt atccatggag atgtggagcc |
| | ttgtcaccaa cctctaactg |
| 661 | cagaactggg atgtggagct ggaagtgcct cctcttctgg |
| | gctgtgctgg tcacagccac |
| 721 | actctgcacc gctaggccgt ccccgacctt gcctgaacaa |
| | gcccagccct ggggagcccc |
| 781 | tgtggaagtg gagtccttcc tggtccaccc cggtgacctg |
| | ctgcagcttc gctgtcggct |
| 841 | gcgggacgat gtgcagagca tcaactggct gcgggacggg |
| | gtgcagctgg cggaaagcaa |
| 901 | ccgcacccgc atcacagggg aggaggtgga ggtgcaggac |
| | tccgtgcccg cagactccgg |
| 961 | cctctatgct tgcgtaacca gcagcccctc gggcagtgac |
| | accacctact tctccgtcaa |
| 1021 | tgtttcagat gctctcccct cctcggagga tgatgatgat |
| | gatgatgact cctcttcaga |
| 1081 | ggagaaagaa acagataaca ccaaaccaaa ccgtatgccc |
| | gtagctccat attggacatc |
| 1141 | cccagaaaag atggaaaaga aattgcatgc agtgccggct |
| | gccaagacag tgaagttcaa |
| 1201 | atgcccttcc agtgggaccc caaaccccac actgcgctgg |
| | ttgaaaaatg gcaaagaatt |
| 1261 | caaacctgac cacagaattg gaggctacaa ggtccgttat |
| | gccacctgga gcatcataat |
| 1321 | ggactctgtg gtgccctctg acaagggcaa ctacacctgc |
| | attgtggaga atgagtacgg |
| 1381 | cagcatcaac cacacatacc agctggatgt cgtggagcgg |
| | tcccctcacc ggcccatcct |
| 1441 | gcaagcaggg ttgcccgcca acaaaacagt ggccctgggt |
| | agcaacgtgg agttcatgtg |
| 1501 | taaggtgtac agtgacccgc agccgcacat ccagtggcta |
| | aagcacatcg aggtgaatgg |
| 1561 | gagcaagatt ggcccagaca acctgcctta tgtccagatc |
| | ttgaagactg ctggagttaa |
| 1621 | taccaccgac aaagagatgg aggtgcttca cttaagaaat |
| | gtctcctttg aggacgcagg |
| 1681 | ggagtatacg tgcttggcgg gtaactctat cggactctcc |
| | catcactctg catggttgac |
| 1741 | cgttctggaa gccctggaag agaggccggc agtgatgacc |
| | tcgcccctgt acctggagat |
| 1801 | catcatctat tgcacagggg ccttcctcat ctcctgcatg |
| | gtggggtcgg tcatcgtcta |
| 1861 | caagatgaag agtggtacca agaagagtga cttccacagc |
| | cagatggctg tgcacaagct |
| 1921 | ggccaagagc atccctctgc gcagacaggt gtctgctgac |
| | tccagtgcat ccatgaactc |
| 1981 | tggggttctt ctggttcggc catcacggct ctcctccagt |
| | gggactccca tgctagcagg |
| 2041 | ggtctctgag tatgagcttc ccgaagaccc tcgctgggag |
| | ctgcctcggg acagactggt |
| 2101 | cttaggcaaa cccctgggag agggctgctt tgggcaggtg |
| | gtgttggcag aggctatcgg |
| 2161 | gctggacaag gacaaaccca accgtgtgac caaagtggct |
| | gtgaagatgt tgaagtcgga |
| 2221 | cgcaacagag aaagacttgt cagacctgat ctcagaaatg |
| | gagatgatga agatgatcgg |
| 2281 | gaagcataag aatatcatca acctgctggg ggcctgcacg |
| | caggatggtc ccttgtatgt |
| 2341 | catcgtggag tatgcctcca agggcaacct gcgggagtac |
| | ctgcaggccc ggaggccccc |
| 2401 | agggctggaa tactgctaca accccagcca caacccagag |
| | gagcagctct cctccaagga |
| 2461 | cctggtgtcc tgcgcctacc aggtggcccg aggcatggag |
| | tatctggcct ccaagaagtg |
| 2521 | catacaccga gacctggcag ccaggaatgt cctggtgaca |
| | gaggacaatg tgatgaagat |
| 2581 | agcagacttt ggcctcgcac gggacattca ccacatcgac |
| | tactataaaa agacaaccaa |
| 2641 | cggccgactg cctgtgaagt ggatggcacc cgaggcatta |
| | tttgaccgga tctacaccca |
| 2701 | ccagagtgat gtgtggtctt tcggggtgct cctgtgggag |
| | atcttcactc tgggcggctc |
| 2761 | cccatacccc ggtgtgcctg tggaggaact tttcaagctg |
| | ctgaaggagg gtcaccgcat |
| 2821 | ggacaagccc agtaactgca ccaacgagct gtacatgatg |
| | atgcgggact gctggcatgc |
| 2881 | agtgccctca cagagaccca ccttcaagca gctggtggaa |
| | gacctggacc gcatcgtggc |
| 2941 | cttgacctcc aaccaggagt acctggacct gtccatgccc |
| | ctggaccagt actcccccag |
| 3001 | ctttcccgac acccggagct ctacgtgctc ctcaggggag |
| | gattccgtct tctctcatga |
| 3061 | gccgctgccc gaggagccct gcctgccccg acacccagcc |
| | cagcttgcca atggcggact |
| 3121 | caaacgccgc tgactgccac ccacacgccc tccccagact |
| | ccaccgtcag ctgtaaccct |
| 3181 | cacccacagc ccctgctggg cccaccacct gtccgtccct |
| | gtcccctttc ctgctggcag |
| 3241 | gagccggctg cctaccaggg gccttcctgt gtggcctgcc |
| | ttcaccccac tcagctcacc |
| 3301 | tctccctcca cctcctctcc acctgctggt gagaggtgca |
| | aagaggcaga tctttgctgc |
| 3361 | cagccacttc atcccctccc agatgttgga ccaacacccc |
| | tccctgccac caggcactgc |
| 3421 | ctggagggca gggagtggga gccaatgaac aggcatgcaa |
| | gtgagagctt cctgagcttt |
| 3481 | ctcctgtcgg tttggtctgt tttgccttca cccataagcc |
| | cctcgcactc tggtggcagg |
| 3541 | tgccttgtcc tcagggctac agcagtaggg aggtcagtgc |
| | ttcgtgcctc gattgaaggt |
| 3601 | gacctctgcc ccagataggt ggtgccagtg gcttattaat |
| | tccgatacta gtttgctttg |
| 3661 | ctgaccaaat gcctggtacc agaggatggt gaggcgaagg |
| | ccaggttggg ggcagtgttg |
| 3721 | tggccctggg gcccagcccc aaactggggg ctctgtatat |
| | agctatgaag aaaacacaaa |
| 3781 | gtgtataaat ctgagtatat atttacatgt ctttttaaaa |
| | gggtcgttac cagagattta |
| 3841 | cccatcgggt aagatgctcc tggtggctgg gaggcatcag |
| | ttgctatata ttaaaaacaa |
| 3901 | aaaaaaaaaa aaa |

By “FRS2α polypeptide” is meant a polypeptide or fragment thereof having at least about 85% amino acid identity to NCBI Accession No. NP_001265286.1 and having a biological activity of a FRS2α polypeptide. Biological activities of a FRS2α polypeptide include transmembrane receptor protein tyrosine kinase adaptor activity and binding to a FGFR1 polypeptide. The sequence at NCBI Accession No. NP_001265286.1 is shown below (SEQ ID No: 5):

| | |
|---|---|
| 1 | mgsccscpdk dtvpdnhrnk fkvinvdddg nelgsgimel tdtelilytr krdsvkwhyl |
| 61 | clrrygydsn lfsfesgrrc qtgqgifafk caraeelfnm lqeimqnnsi nvveepvver |
| 121 | nnhqtelevp rtprtpttpg faaqnlpngy prypsfgdas shpssrhpsv gsarlpsvge |
| 181 | esthpllvae eqvhtyvntt gvqeerknrt svhvplearv snaesstpke epssiedrdp |
| 241 | qillepegvk fvlgptpvqk qlmekekleq lgrdqvsgsg anntewdtgy dsderrdaps |
| 301 | vnklvyenin glsipsasgv rrgrltstst sdtqninnsa qrrtallnye nlpslppvwe |
| 361 | arklsrdedd nlgpktpsln gyhnnldpmh nyvntenvtv pasahkieys rrrdctptvf |
| 421 | nfdirrpsle hrqlnyiqvd leggsdsdnp qtpktpttpl pqtptrrtel yavidierta |
| 481 | amsnlqkalp rddgtsrktr hnstdlpm |

By “FRS2α polynucleotide” is meant a polynucleotide encoding a FRS2α polypeptide. An exemplary FRS2α polynucleotide sequence is provided at NCBI Accession No. NM_001278357.1. The exemplary sequence provided at NCBI Accession No. NM_001278357.1 is reproduced below (SEQ ID No: 6).

| | |
|---|---|
| 1 | aaaacccttc cctcccccgc tcccccggaa gtgcttttcc aagattcggg ccggagagag |
| 61 | gccttgtagg cacagcggct gagactcgat ctgctccaag taggggctcc agcgcgggtc |
| 121 | ggagtctggg ggttcgcgcc cgccgacccg cgccctgctc cctctcagca cctgggcgga |
| 181 | cggttaaatc agcaaacaaa gaaaacatgg tattttgaaa tatgattaaa ctcctgatgc |
| 241 | tgcagcagag gctaagaata ttaatggcca gatctagtgc acacatggtc ttctgaagaa |
| 301 | gccatgggta gctgttgtag ctgtccagat aaagacactg tcccagataa ccatcggaac |
| 361 | aagtttaagg tcattaatgt ggatgatgat gggaatgagt taggttctgg cataatggaa |
| 421 | cttacagaca cagaactgat tttatacacc cgcaaacgtg actcagtaaa atggcactac |
| 481 | ctctgcctgc gacgctatgg ctatgactcg aatctctttt cttttgaaag tggtcgaagg |
| 541 | tgtcaaactg gacaaggaat ctttgccttt aagtgtgccc gtgcagaaga attatttaac |
| 601 | atgttgcaag agattatgca aaataatagt ataaatgtgg tggaagagcc agttgtagaa |
| 661 | agaaataatc atcagacaga attggaagtc cctagaacac ctcgaacacc tacaactcca |
| 721 | ggatttgctg ctcagaactt acctaatgga tatccccgat atccctcatt tggagatgct |
| 781 | tcatcccatc cgtcaagcag acatccttct gtgggaagtg ctcgcctgcc ttcagtaggg |
| 841 | gaagaatcta cacatccttt gcttgtggct gaggaacaag tacataccta tgtcaacact |
| 901 | acaggtgtgc aagaagagcg gaaaaaccgc acaagtgtgc atgttccatt ggaggcgagg |
| 961 | gtttctaacg ctgaaagcag cacaccaaaa gaagaaccaa gtagtattga ggacagggat |
| 1021 | cctcagattc ttcttgaacc tgaaggagtc aaatttgttt tagggccaac ccctgttcaa |
| 1081 | aagcagttaa tggaaaaaga gaaactggag caacttggaa gagatcaagt tagtggaagt |
| 1141 | ggagcaaata acacagaatg ggacactggc tatgacagtg atgaacgaag agatgcaccc |
| 1201 | tctgttaaca aactggtgta tgaaaatata aatgggctat ctatccctag tgcctcaggg |
| 1261 | gtcaggagag gtcgtctgac atccaccagt acctcagata cccagaatat caacaactca |
| 1321 | gctcagagaa gaactgcatt attaaactat gaaaatctac catctttgcc tcctgtttgg |
| 1381 | gaagcccgca agctaagtag ggatgaagat gacaatttag gaccaaagac cccatctcta |
| 1441 | aatggctacc ataataatct agatccaatg cataactatg taaatacaga gaatgtaaca |
| 1501 | gtgccagcaa gtgctcacaa aatagaatat tcaaggcgtc gggactgtac accaacagtc |
| 1561 | tttaactttg atatcagacg cccaagttta gaacacaggc agcttaatta catacaggtt |
| 1621 | gacttggaag gtggcagtga ctctgacaac cctcagactc caaaaacgcc tacaactccc |
| 1681 | cttccacaaa cccctaccag gcgcacagag ctgtatgccg tgatagacat cgagagaact |
| 1741 | gctgctatgt caaatttgca gaaagcactg ccacgagatg atggtacatc taggaaaact |
| 1801 | agacacaata gtactgatct gcccatgtga gcctggaaag cattgtgttg tttgcacctt |
| 1861 | tgtgaagttt ttaaaaatga agatgcaagt gcttcatttt catttctaaa cactaactcc |
| 1921 | ttttatagac tgataaaatt tttttctgaa tatttcatgt gcatctttaa ctaaagggaa |
| 1981 | ttaatgtaga gcaggtactc cttaaagaac actaatttca ttatatacta ctcgttgtac |
| 2041 | agcagcattc ccgttttcac agtgcctatt taaaatgaga gttgaagtaa atgacatgct |
| 2101 | ggttgatttt tatcaatatt ctggacttaa cgcatacctt tcatgtctaa gtcatggttg |
| 2161 | gcttttaaaa ctttttataa agcctcttga caatgtacat tgctaacagg taactatagg |
| 2221 | ctttgaaagt aatgctcgta gattcagtgt tcacagtatg tggcctccag catgtaacat |
| 2281 | gaggaatcct ttatttcatt aattaatggc tttttgactt gagccaaaac atatgtaaag |
| 2341 | gaaacagaag taccgcacct cctcttacac cagtcagctc ctttgccttc agtgttacta |
| 2401 | gaaagcggcc tgtgtccatg agtgtgcttt gctgttggtg cactgaaagg caggaaggag |
| 2461 | acaagatttt ctatttactc atctcatgat gtcatttgaa gggcatgtcc agatatctta |
| 2521 | aaattataat aggctcaaga atcagtctca ggtcacttta cccaaaaaca tttgaaaatc |
| 2581 | tgaaccacaa tctcctgaaa gtttttctcc tatagattgt tgacaacaca ttgttttctg |
| 2641 | gaggcatttg tgccattagg tttccattta tcttcagttt ttttctttgg tgtttgggat |
| 2701 | gtcttatttt gttgccttat gtccttttca atttaaaatg tttgagtttg tatatagttt |
| 2761 | tgaaattgga ttatgtgttc attgttgttt agtttgcatt tttgtcaaat tatggttttg |
| 2821 | aaggttcatt tggaacttac tgttagtctg taacagggtt gcccttgtcc agtatttatt |
| 2881 | tataagctgt ttacttttca agttgataaa aacattctcc aattctaaat ttgcttgtgt |
| 2941 | ccataggtga tctctttagc aaactgagaa aaaaaggaag ctacttttaa catgcaaagt |
| 3001 | tccctcaagg tgtaccgtgt tgtctctgtg ggcactcttc cccagcactt tagcagtaat |
| 3061 | tcccccagct acacgctgca gttgtactct gcccactcta gtgttcctca gctctgctgt |
| 3121 | ccttttactt gtagctggat ctttgattat ccttcgattt ccatgaaata ttaatattgt |
| 3181 | tgccagcata gcaggtacag tggaagtctt gtagcagtga gattgtatca taatttagga |
| 3241 | tttaaaatga attaaagttt atataaactg aagagtctcc atatgtcaaa ctcttggaaa |
| 3301 | atcaaagatg ttccaatttc ctaaacacta gagaatacga gagaaggtag agtggaaaag |
| 3361 | gttaggtaac cttgcaaaat attttactat tttctctaaa tatgaggaag tttgagatta |
| 3421 | tgatctggat ctaccagata taactaaggt taatttagca tgaaaaagtt ttagtcatat |
| 3481 | tggcatccaa cctattcagt aaccgaatca taggacaatg atggattagg agaacaatag |
| 3541 | agtgggatca ttataaagaa aataaattat taaaggtgtc tttatcgttt tagtgccatt |
| 3601 | tttagtgtct ttactataaa tcaatatcag tgtattttat cattctatgt gcatagcaga |
| 3661 | attttctttt ctcccttttg ttcccctgtg aacttggtgc ttattaaagt gctcactgtt |
| 3721 | ctcttaaaag agagcagtgg tataggtgtg cagtttccat gatgcaggtt ccatttttaa |
| 3781 | tatattgttc cacttatcct ttcttctgag taaattgcta attgtgccaa atttatgtaa |
| 3841 | tagtttttgt aatgtggaat aagaattatg atggaaccat tgcacatttt tttctgaaac |
| 3901 | agccagtcaa ggcagaacat taatctccaa atgcaagggc tgatctattt attcattttg |
| 3961 | gaggttgggt actttattct ttctttccgt catccttttc attgtttccc ccggattcta |
| 4021 | attagttttt atttttttta gataactcca atataatcat tacagtttat gctttaaata |
| 4081 | ctatgtgctt taaaaaggaa aatgggacca atttgtctgc taagaatttg attttaggta |
| 4141 | ctataagagt attaggaaaa tatatacaac tggtgttaat ttctagatat tttctagaaa |
| 4201 | tcacttgtgt tcctatttaa taaaaggtaa tttagaatac tacttgtcct ttgcagtagt |
| 4261 | ttagtaatgg gcattaagct gtgtcctcga aggatgtacc tattactagg tgcattttag |
| 4321 | aatgaaatat tgatatttta ttagcatata attgtggcca tatatctcag attttctgag |
| 4381 | gcagatctaa ttttagataa ttctgttggt agaccatgtg atccttcttt ttggttttgg |
| 4441 | aaatataatc attgttaatg ttttccctcc aaatagaata ctgttttatc catacaaatc |
| 4501 | ataacagcat ctatcccatg ctagggttgg aaactgatat tggtattact tgtgtttttt |
| 4561 | cttagtgtgt tttatttccc agtttcatct tcttctaaaa atgaaaatat ggtgccttcc |
| 4621 | ctccctccag gaagactggc aaatatttcc ttttatttac tgctgctgtg gagtgatgag |
| 4681 | atatgcactt tactctttaa gattcagcaa aaagcttttc acttctcagt atatccagaa |
| 4741 | tacatcatat ctgggactta ggaaaatttg ccaagcaatc tttgttttta tagatactaa |
| 4801 | tgttgaccct ctccagcgtt caatgttata aatagaacaa gtcaagctag tgtttatctc |
| 4861 | ctccccctcc ccaaaactgt ggcacagcat ataaaaatgt acctcaataa tgttctatta |
| 4921 | aaaatgggac aggggcctta tgttttcata atttcccaac aatgtgccgc catatttttg |
| 4981 | cctcaaggta aaggttttaa cagatgaaaa agtacttccc aattcccccg tgctattcct |
| 5041 | aacctataat gcccaaatgt tttgtgcaat gtgtagtgtg tgtgtataaa tacatatatt |
| 5101 | cttgaaatag acataccatc agagacatca ttcacaagta actgatgtat tggcatctca |
| 5161 | ttcatatttc tgatgtgtga ggtatatggt actaattacc ttttccttga tgtttgccaa |
| 5221 | atttgaataa aggcattggt acgaaattac agaatgtaaa gaaaatgttt ttggcttgaa |
| 5281 | aaattaacat attttatgac gtaccacagt atactctgcc caaaccagca ccctatctat |
| 5341 | ctttcctgtt ctttacatcc ctgttcccca tccctacttc ctcatttttg gtataacaca |
| 5401 | gttcttttgt agcatcatta taattgcagt tctatggcaa ttggacagtt atagcatgga |
| 5461 | aacagactgg tataagtagt acagtagtca ccagtgtgcc acatttgcat tagtaatgca |
| 5521 | aaatatacat tttataaagg acaaactttg tgttatgttt tattttcatt acattgtata |
| 5581 | atattgtaag actattgtat gtcctaattt gcattataaa tgtttttttc ctacgtaaag |
| 5641 | gcataaatat agcaactttg tataaaggta gcttattaga tttttaattt tttcttttat |
| 5701 | aaaaaattgt ccaacagtgg gactaccatt gccaaattgt atatgaaata tgaattttac |
| 5761 | ccccatggtt aatttctttt ataaacattc catatttctc taataaaaag acataagtga |
| 5821 | tactgtacta tgcatacatt gtatcttaat gctgtttcag atcagcattt taaattttgg |
| 5881 | tttgcatttt taatattggc aaaacgtaac cactgttaat taaaataaaa ccttgttgta |
| 5941 | tatgtaacaa cataattttc cctctatccc ttcccaccct ttgttctcta tttctcccta |
| 6001 | tcagtgccaa cttcatacat tttgtagcat ggcaataaaa tataactttt acactgaggc |
| 6061 | cgagtgtggc tttttggagg aagtggggat gggacgattg ccctctagtt gtcctttgca |
| 6121 | tatgactgtt ttttgccata taagccatgt catcaggcat gaaaagtttt ctcatatatg |
| 6181 | atgtaaactt gcttttaagg acaagtgtga atgtgctttt taagcttaat ttttgtcatg |
| 6241 | acaactaatt ttttttatct ttggagaagt cagagttctt tacaatcaaa cgtttattaa |
| 6301 | ctggagtact tagaataagc tagtaattga atttagttca agggctaagc aacacatttt |
| 6361 | taaatcctta tttattgtag agtattagta tactgtccta caaattatgt aaaatatggt |
| 6421 | ttaatattag atgactttgg attttgcaat gccttactgt tgtcattcta gcataaatat |
| 6481 | ccataatgag gtactcaagt tgatactgga agctgagctg atcatacact gacctgaagc |
| 6541 | attcatgaaa agctgcttta ttgaataaag tctgattgga gttcttttca tgctcacttt |
| 6601 | ccccttattg ctgaaagtag attgcaataa aaccccaata aaacgtttgg tcggatatct |
| 6661 | acttaaaaaa aaaaaa |

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any polypeptide or polynucleotide having an alteration in expression level, sequence, or activity that is associated with a disease or disorder or risk of disease or disorder. In some embodiments, a decrease in activity or level of a FGF signaling polypeptide or let-7 miRNA in an endothelial cell is associated with development and/or progression of PAH. In some embodiments, an increase in level or activity of a TGFβ signaling polypeptide (e.g., TGFβ1, TGFβ2, TGFβ3, TGFβR1, TGFβR2) in an endothelial cell is associated with development and/or progression of PAH. In some other embodiments, an increase in activity or level of a FGF signaling polypeptide or let-7 miRNA in a smooth muscle cell is associated with development and/or progression of PAH. In still other embodiments, a decrease in level or activity of a TGFβ signaling polypeptide (e.g., TGFβ1, TGFβ2, TGFβ3, TGFβR1, TGFβR2) is associated with development and/or progression of PAH.

As used herein, “microRNA” or “miRNA” describes small non-coding RNA molecules, generally about 15 to about 50 nucleotides in length, preferably 17-23 nucleotides, 15 which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from 20 primary transcripts (pri-miRNA) through sequential cleavage by RNAse III enzymes. miRBase is a comprehensive microRNA database located at www.mirbase.org, incorporated by reference herein in its entirety for all purposes.

By “let-7 miRNA” is meant a miRNA member of the let-7 miRNA family. Sequences of members of the let-7 miRNA family can be found in, for example, www.mirbase.org. Exemplary members of the let-7 miRNA family include hsa-let-7b or human let-7b (miRBase Accession No. MI0000063), hsa-let-7a-1 (miRBase Accession No. MI0000060), hsa-let-7a-2 (miRBase Accession No. MI0000061), hsa-let-7a-3 (miRBase Accession No. MI0000062), hsa-let-7b, hsa-let-7c (miRBase Accession No. MI0000064), hsa-let-7d (miRBase Accession No. MI0000065), hsa-let-7e (miRBase Accession No. MI0000066), hsa-let-7f-1 (miRBase Accession No. MI0000067), hsa-let-7f-2 (miRBase Accession No. MI0000068), hsa-let-7g (miRBase Accession No. MI0000433), and hsa-let-7i (miRBase Accession No. MI00000434). The sequence of human let-7b provided at miRBase Accession No. MI0000063 is reproduced below.

human let-7b (5 prime):
(SEQ ID No: 1)
UGAGGUAGUAGGUUGUGUGGUU
human let-7b (3 prime):
(SEQ ID No: 2)
CUAUACAACCUACUGCCUUCCC

The let-7 miRNA family has been shown to play important roles in animal development, cell differentiation, and metabolism. In some embodiments, an activity of let-7 miRNA is repression of expression of a TGFβ signaling polypeptide. In some embodiments, an activity of let-7 miRNA is repression of TGFβ signaling.

In some embodiments, the let-7 miRNA is used as a therapeutic. Use of let-7 miRNA as a therapeutic has been demonstrated previously. For example, let-7 miRNA was used as anti-cancer therapy (Trang et al., Mol Ther. 2011 June; 19(6): 1116-1122).

In some embodiments, the let-7 miRNA is chemically modified. In particular embodiments, uracil (“U”) or cytosine (“C”) is chemically modified. In some embodiments, the miRNA is modified to impart properties to the miRNA to make it useful as a therapeutic, such as attenuated immunostimulation and increased serum stability. Such modifications to the miRNA include, without limitation, incorporation of a 2′-O-methyl (2′-O-Me), phosphorothioate (PS), and deoxy thymidine (dT) residues. In particular embodiments, the modified miRNA retains silencing activity in vivo. In particular embodiments, the modification is a 2′-O-methyl nucleotide modification. In some embodiments, the modification decreases the likelihood of triggering an innate immune response.

In some embodiments, the let-7 miRNA contains a “light” modification. By a miRNA containing a “light modification” is meant that the miRNA contains a 2′-O-methyl modification on all U and C nucleotide bases followed by adenosine (“A”) on the antisense strand. In some other embodiments, the let-7 miRNA contains a “heavy” modification. By a miRNA containing a “heavy modification” is meant that the miRNA contains a 2′-O-methyl modification on all U and C nucleotide bases on the sense strand.

In still other embodiments, the let-7 miRNA is “mi-let-7b_{L}”. mi-let-7b_{L} is also referred to herein as “let-7 light.” The sequence of mi-let-7b_{L} is provided below:

mi-let-7b_{L} (5 prime):
(SEQ ID No: 1)
UGAGGuAGuAGGUUGUGUGGUU
mi-let-7b_{L} (3 prime):
(SEQ ID NO: 2)
CuAuAcAACCuACUGCCUUCCC

In some other embodiments, the let-7 miRNA is “mi-let-7b_{H}”. mi-let-7b_{H} is also referred to herein as “let-7 heavy.” The sequence of mi-let-7b_{H} miRNA is provided below:

mi-let-7b_{H} (5 prime):
(SEQ ID No: 1)
UGAGGuAGuAGGUUGUGUGGUU
mi-let-7b_{H} (3 prime):
(SEQ ID NO: 2)
cuAuAcAAccuAcuGccuuccc

In the foregoing sequences, lower case indicates a nucleotide base containing a 2′-O-methyl modification.

As used herein, “obtaining” as in “obtaining an agent” includes synthesizing, purchasing, or otherwise acquiring the agent.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, “polynucleotide” includes cDNA, RNA, DNA/RNA hybrid, antisense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the term “promoter” or “regulatory sequence” means a nucleic acid sequence which is required for expression of a gene product operably linked to the promoter or regulator sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in an inducible manner.

By “pulmonary arterial hypertension” or “PAH” is mean a disease syndrome characterized by increased systolic pressure in the pulmonary artery that exceeds, at rest, 25 mm Hg. This can be due to primary changes in the lung (primary pulmonary hypertension) or secondary to increase left-side cardiac pressures (secondary pulmonary hypertension).

By “reduces” is meant a negative alteration of at least 10%, 25%, 50%, 75%, or 100%.

By “reference” is meant a standard or control condition. In some embodiments, the reference is an activity or level of a TGFβ signaling polypeptide or polynucleotide or a FGF signaling polypeptide or polynucleotide in a healthy, normal subject or in a subject that does not have PAH. In some embodiments, the reference is an activity or level of a let-7 miRNA in a healthy, normal subject or in a subject that does not have PAH. In some embodiments, the TGFβ signaling polypeptide or polynucleotide is a TGFβ1, TGFβ2, TGFβ3, TGFβR1, or TGFβR2 polypeptide or polynucleotide. In some embodiments, the FGF signaling polypeptide is FRS2α. In some other embodiments, the let-7 miNA is at least one selected from the group consisting of human let-7b miRNA and human let-7c miRNA.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant an agent that recognizes and binds a polypeptide or polynucleotide of the invention, but which does not substantially recognize and bind other molecules in a sample, for example, a biological sample, which naturally includes a polynucleotide of the invention. In some embodiments, the agent is a nucleic acid molecule.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, a “TGFβ signaling polypeptide” refers to a member or component of a transformation growth factor β (TGFβ) signaling pathway. Exemplary TGFβ signaling polypeptides include polypeptides TGFβ1, TGFβ2, TGFβ3, TGFβR1, TGFβR2, SMAD1, SMAD2, SMAD3, SMAD4, SMAD5, and SMAD9.

As used herein, a “TGFβ signaling polynucleotide” is a polynucleotide encoding a TGFβ signaling polypeptide.

By “TGFβ1 polypeptide” is meant a polypeptide or fragment thereof having at least about 85% amino acid identity to GenBank Accession No. AAH22242.1 and having a biological activity of a TGFβ1 polypeptide. Biological activities of a TGFβ1 polypeptide include binding to a type II transforming growth factor β (TGFβ) receptor and homodimerization. The sequence at GenBank Accession No. AAH22242.1 is shown below (SEQ ID NO: 7):

| | |
|---|---|
| 1 | mppsglrlll lllpllwllv ltpgrpaagl stcktidmel vkrkrieair gqilsklrla |
| 61 | sppsqgevpp gplpeavlal ynstrdrvag esaepepepe adyyakevtr vlmvethnei |
| 121 | ydkfkqsths iymffntsel reavpepvll sraelrllrl klkveqhvel yqkysnnswr |
| 181 | ylsnrllaps dspewlsfdv tgvvrqwlsr ggeiegfrls ahcscdsrdn tlqvdingft |
| 241 | tgrrgdlati hgmnrpflll matpleraqh lqssrhrral dtnycfsste knccvrglyi |
| 301 | dfrkdlgwkw ihepkgyhan fclgpcpyiw sldtqyskvl alynqhnpga saapccvpqa |
| 361 | leplpivyyv grkpkveqls nmivrsckcs |

By “TGFβ1 polynucleotide” is meant a polynucleotide encoding a TGFβ1 polypeptide. An exemplary TGFβ1 polynucleotide sequence is provided at GenBank Accession No. BC022242.1. The exemplary sequence provided at GenBank Accession No. BC022242.1 is reproduced below (SEQ ID NO: 8).

| | |
|---|---|
| 1 | cccagacctc gggcgcaccc cctgcacgcc gccttcatcc ccggcctgtc tcctgagccc |
| 61 | ccgcgcatcc tagacccttt ctcctccagg agacggatct ctctccgacc tgccacagat |
| 121 | cccctattca agaccaccca ccttctggta ccagatcgcg cccatctagg ttatttccgt |
| 181 | gggatactga gacacccccg gtccaagcct cccctccacc actgcgccct tctccctgag |
| 241 | gacctcagct ttccctcgag gccctcctac cttttgccgg gagaccccca gcccctgcag |
| 301 | gggcggggcc tccccaccac accagccctg ttcgcgctct cggcagtgcc ggggggcgcc |
| 361 | gcctccccca tgccgccctc cgggctgcgg ctgctgctgc tgctgctacc gctgctgtgg |
| 421 | ctactggtgc tgacgcctgg ccggccggcc gcgggactat ccacctgcaa gactatcgac |
| 481 | atggagctgg tgaagcggaa gcgcatcgag gccatccgcg gccagatcct gtccaagctg |
| 541 | cggctcgcca gccccccgag ccagggggag gtgccgcccg gcccgctgcc cgaggccgtg |
| 601 | ctcgccctgt acaacagcac ccgcgaccgg gtggccgggg agagtgcaga accggagccc |
| 661 | gagcctgagg ccgactacta cgccaaggag gtcacccgcg tgctaatggt ggaaacccac |
| 721 | aacgaaatct atgacaagtt caagcagagt acacacagca tatatatgtt cttcaacaca |
| 781 | tcagagctcc gagaagcggt acctgaaccc gtgttgctct cccgggcaga gctgcgtctg |
| 841 | ctgaggctca agttaaaagt ggagcagcac gtggagctgt accagaaata cagcaacaat |
| 901 | tcctggcgat acctcagcaa ccggctgctg gcacccagcg actcgccaga gtggttatct |
| 961 | tttgatgtca ccggagttgt gcggcagtgg ttgagccgtg gaggggaaat tgagggcttt |
| 1021 | cgccttagcg cccactgctc ctgtgacagc agggataaca cactgcaagt ggacatcaac |
| 1081 | gggttcacta ccggccgccg aggtgacctg gccaccattc atggcatgaa ccggcctttc |
| 1141 | ctgcttctca tggccacccc gctggagagg gcccagcatc tgcaaagctc ccggcaccgc |
| 1201 | cgagccctgg acaccaacta ttgcttcagc tccacggaga agaactgctg cgtgcggcag |
| 1261 | ctgtacattg acttccgcaa ggacctcggc tggaagtgga tccacgagcc caagggctac |
| 1321 | catgccaact tctgcctcgg gccctgcccc tacatttgga gcctggacac gcagtacagc |
| 1381 | aaggtcctgg ccctgtacaa ccagcataac ccgggcgcct cggcggcgcc gtgctgcgtg |
| 1441 | ccgcaggcgc tggagccgct gcccatcgtg tactacgtgg gccgcaagcc caaggtggag |
| 1501 | cagctgtcca acatgatcgt gcgctcctgc aagtgcagct gaggtcccgc cccgccccgc |
| 1561 | cccgccccgg caggcccggc cccaccccgc cccgcccccg ctgccttgcc catgggggct |
| 1621 | gtatttaagg acacccgtgc cccaagccca cctggggccc cattaaagat ggagagagga |
| 1681 | aaaaaaaaaa aaaaaaaaaa aaaaaaaaaa aaaaaaaaaa aaaaaaaaaa aaaaaaaaaa |
| 1741 | aaaaaa |

By “TGFβ2 polypeptide” is meant a polypeptide or fragment thereof having at least about 85% amino acid identity to GenBank Accession No. AAA50405.1 and having a biological activity of a TGFβ2 polypeptide. Biological activities of a TGFβ2 polypeptide include binding to a type II transforming growth factor β (TGFβ) receptor and homodimerization. The sequence at GenBank Accession No. AAA50405.1 is shown below (SEQ ID NO: 9):

| | |
|---|---|
| 1 | mhycvlsafl ilhlvtvals lstcstldmd qfmrkrieai rgqilsklkl tsppedypep |
| 61 | eevppevisi ynstrdllqe kasrraaace rersdeeyya kevykidmpp ffpseaippt |
| 121 | fyrpyfrivr fdvsamekna snlvkaefrv frlqnpkarv peqrielyqi lkskdltspt |
| 181 | qryidskvvk traegewlsf dvtdavhewl hhkdrnlgfk islhcpcctf vpsnnyiipn |
| 241 | kseelearfa gidgtstyts gdqktikstr kknsgktphl llmllpsyrl esqqtnrrkk |
| 301 | raldaaycfr nvqdncclrp lyidfkrdlg wkwihepkgy nanfcagacp ylwssdtqhs |
| 361 | rvlslyntin peasaspccv sqdlepltil yyigktpkie qlsnmivksc kcs |

By “TGFβ2 polynucleotide” is meant a polynucleotide encoding a TGFβ2 polypeptide. An exemplary TGFβ2 polynucleotide sequence is provided at GenBank Accession No. M19154.1. The exemplary sequence provided at GenBank Accession No. M19154.1 is reproduced below (SEQ ID NO: 10).

| | |
|---|---|
| 1 | gcccctcccg tcagttcgcc agctgccagc cccgggacct tttcatctct tcccttttgg |
| 61 | ccggaggagc cgagttcaga tccgccactc cgcacccgag actgacacac tgaactccac |
| 121 | ttcctcctct taaatttatt tctacttaat agccactcgt ctcttttttt ccccatctca |
| 181 | ttgctccaag aatttttttc ttcttactcg ccaaagtcag ggttccctct gcccgtcccg |
| 241 | tattaatatt tccacttttg gaactactgg ccttttcttt ttaaaggaat tcaagcagga |
| 301 | tacgtttttc tgttgggcat tgactagatt gtttgcaaaa gtttcgcatc aaaaacaaca |
| 361 | acaacaaaaa accaaacaac tctccttgat ctatactttg agaattgttg atttcttttt |
| 421 | tttattctga cttttaaaaa caactttttt ttccactttt ttaaaaaatg cactactgtg |
| 481 | tgctgagcgc ttttctgatc ctgcatctgg tcacggtcgc gctcagcctg tctacctgca |
| 541 | gcacactcga tatggaccag ttcatgcgca agaggatcga ggcgatccgc gggcagatcc |
| 601 | tgagcaagct gaagctcacc agtcccccag aagactatcc tgagcccgag gaagtccccc |
| 661 | cggaggtgat ttccatctac aacagcacca gggacttgct ccaggagaag gcgagccgga |
| 721 | gggcggccgc ctgcgagcgc gagaggagcg acgaagagta ctacgccaag gaggtttaca |
| 781 | aaatagacat gccgcccttc ttcccctccg aaactgtctg cccagttgtt acaacaccct |
| 841 | ctggctcagt gggcagcttg tgctccagac agtcccaggt gctctgtggg taccttgatg |
| 901 | ccatcccgcc cactttctac agaccctact tcagaattgt tcgatttgac gtctcagcaa |
| 961 | tggagaagaa tgcttccaat ttggtgaaag cagagttcag agtctttcgt ttgcagaacc |
| 1021 | caaaagccag agtgcctgaa caacggattg agctatatca gattctcaag tccaaagatt |
| 1081 | taacatctcc aacccagcgc tacatcgaca gcaaagttgt gaaaacaaga gcagaaggcg |
| 1141 | aatggctctc cttcgatgta actgatgctg ttcatgaatg gcttcaccat aaagacagga |
| 1201 | acctgggatt taaaataagc ttacactgtc cctgctgcac ttttgtacca tctaataatt |
| 1261 | acatcatccc aaataaaagt gaagaactag aagcaagatt tgcaggtatt gatggcacct |
| 1321 | ccacatatac cagtggtgat cagaaaacta taaagtccac taggaaaaaa aacagtggga |
| 1381 | agaccccaca tctcctgcta atgttattgc cctcctacag acttgagtca caacagacca |
| 1441 | accggcggaa gaagcgtgct ttggatgcgg cctattgctt tagaaatgtg caggataatt |
| 1501 | gctgcctacg tccactttac attgatttca agagggatct agggtggaaa tggatacacg |
| 1561 | aacccaaagg gtacaatgcc aacttctgtg ctggagcatg cccgtattta tggagttcag |
| 1621 | acactcagca cagcagggtc ctgagcttat ataataccat aaatccagaa gcatctgctt |
| 1681 | ctccttgctg cgtgtcccaa gatttagaac ctctaaccat tctctactac attggcaaaa |
| 1741 | cacccaagat tgaacagctt tctaatatga ttgtaaagtc ttgcaaatgc agctaaaatt |
| 1801 | cttggaaaag tggcaagacc aaaatgacaa tgatgatgat aatgatgatg acgacgacaa |
| 1861 | cgatgatgct tgtaacaaga aaacataaga gagccttggt tcatcagtgt taaaaaattt |
| 1921 | ttgaaaaggc ggtactagtt cagacacttt ggaagtttgt gttctgtttg ttaaaactgg |
| 1981 | catctgacac aaaaaaagtt gaaggcctta ttctacattt cacctacttt gtaagtgaga |
| 2041 | gagacaagaa gcaaattttt tttaaagaaa aaaataaaca ctggaagaat ttattagtgt |
| 2101 | taattatgtg aacaacgaca acaacaacaa caacaacaaa caggaaaatc ccattaagtg |
| 2161 | gagttgctgt acgtaccgtt cctatcccgc gcctcacttg atttttctgt attgctatgc |
| 2221 | aataggcacc cttcccattc ttactcttag agttaacagt gagttattta ttgtgtgtta |
| 2281 | ctatataatg aacgtttcat tgcccttgga aaataaaaca ggtgtataaa gtggagacca |
| 2341 | aatactttgc cagaaactca tggatggctt aaggaacttg aactcaaacg agccagaaaa |
| 2401 | aaagaggtca tattaatggg atgaaaaccc aagtgagtta ttatatgacc gagaaagtct |
| 2461 | gcattaagat aaagaccctg aaaacacatg ttatgtatca gctgcctaag gaagcttctt |
| 2521 | gtaaggtcca aaaactaaaa agactgttaa taaaagaaac tttcagtcag |

By “TGFβ3 polypeptide” is meant a polypeptide or fragment thereof having at least about 85% amino acid identity to GenBank Accession No. EAW81249.1 and having a biological activity of a TGFβ3 polypeptide. Biological activities of a TGFβ3 polypeptide include binding to a type II transforming growth factor β (TGFβ) receptor and homodimerization. The sequence at GenBank Accession No. EAW81249.1 is shown below (SEQ ID NO: 11):

| | |
|---|---|
| 1 | mkmhlqralv vlallnfatv slslstcttl dfghikkkrv eairgqilsk lrltsppept |
| 61 | vmthvpyqvl alynstrell eemhgereeg ctqentesey yakeihkfdm iqglaehnel |
| 121 | avcpkgitsk vfrfnvssve knrtnlfrae frvlrvpnps skrnegriel fqilrpdehi |
| 181 | akqryiggkn lptrgtaewl sfdvtdtvre wllrresnlg leisihcpch tfqpngdile |
| 241 | nihevmeikf kgvdneddhg rgdlgrlkkg kdhhnphlil mmipphrldn pgqggqrkkr |
| 301 | aldtnycfrn leenccvrpl yidfrqdlgw kwvhepkgyy anfcsgpcpy lrsadtthst |
| 361 | vlglyntlnp easaspccvp qdlepltily yvgrtpkveq lsnmvvksck cs |

By “TGFβ3 polynucleotide” is meant a polynucleotide encoding a TGFβ3 polypeptide. An exemplary TGFβ3 polynucleotide sequence is provided at NCBI Accession No. NG 011715.1. The exemplary sequence provided at NCBI Accession No. BT007287.1 is reproduced below (SEQ ID NO: 12).

| | |
|---|---|
| 1 | atgaagatgc acttgcaaag ggctctggtg gtcctggccc tgctgaactt tgccacggtc |
| 61 | agcctctctc tgtccacttg caccaccttg gacttcggcc acatcaagaa gaagagggtg |
| 121 | gaagccatta ggggacagat cttgagcaag ctcaggctca ccagcccccc tgagccaacg |
| 181 | gtgatgaccc acgtccccta tcaggtcctg gccctttaca acagcacccg ggagctgctg |
| 241 | gaggagatgc atggggagag ggaggaaggc tgcacccagg aaaacaccga gtcggaatac |
| 301 | tatgccaaag aaatccataa attcgacatg atccaggggc tggcggagca caacgaactg |
| 361 | gctgtctgcc ctaaaggaat tacctccaag gttttccgct tcaatgtgtc ctcagtggag |
| 421 | aaaaatagaa ccaacctatt ccgagcagaa ttccgggtct tgcgggtgcc caaccccagc |
| 481 | tctaagcgga atgagcagag gatcgagctc ttccagatcc ttcggccaga tgagcacatt |
| 541 | gccaaacagc gctatatcgg tggcaagaat ctgcccacac ggggcactgc cgagtggctg |
| 601 | tcctttgatg tcactgacac tgtgcgtgag tggctgttga gaagagagtc caacttaggt |
| 661 | ctagaaatca gcattcactg tccatgtcac acctttcagc ccaatggaga tatcctggaa |
| 721 | aacattcacg aggtgatgga aatcaaattc aaaggcgtgg acaatgagga tgaccatggc |
| 781 | cgtggagatc tggggcgcct caagaagcag aaggatcacc acaaccctca tctaatcctc |
| 841 | atgatgattc ccccacaccg gctcgacaac ccgggccagg ggggtcagag gaagaagcgg |
| 901 | gctttggaca ccaattactg cttccggtag |

By “TGFβR1 polypeptide” is meant a polypeptide or fragment thereof having at least about 85% amino acid identity to GenBank Accession No. AAH71181.1 and having a biological activity of a TGFβR1 polypeptide. Biological activities of a TGFβR1 polypeptide include binding to ligands TGFβ1, TGFβ2, and TGFβ3 polypeptides, and transduction of a signal from TGFβ1, TGFβ2, or TGFβ3 polypeptide binding from the cell surface to the cytoplasm. The sequence at GenBank Accession No. AAH71181.1 is shown below (SEQ ID NO: 13):

| | |
|---|---|
| 1 | meaavaaprp rllllvlaaa aaaaaallpg atalqcfchl ctkdnftcvt dglcfvsvte |
| 61 | ttdkvihnsm ciaeidlipr drpfvcapss ktgsvtttyc cnqdhcnkie lpttglpllv |
| 121 | qrtiartivl qesigkgrfg evwrgkwrge evavkifssr eerswfreae iyqtvmlrhe |
| 181 | nilgfiaadn kdngtwtqlw lvsdyhehgs lfdylnrytv tvegmiklal stasglahlh |
| 241 | meivgtqgkp aiahrdlksk nilvkkngtc ciadlglavr hdsatdtidi apnhrvgtkr |
| 301 | ymapevldds inmkhfesfk radiyamglv fweiarrcsi ggihedyqlp yydlvpsdps |
| 361 | veemrkvvce qklrpnipnr wqscealrvm akimrecwya ngaarltalr ikktlsqlsq |
| 421 | qegikm |

By “TGFβR1 polynucleotide” is meant a polynucleotide encoding a TGFβR1 polypeptide. An exemplary TGFβR1 polynucleotide sequence is provided at GenBank Accession No. BC071181.1. The exemplary sequence provided at GenBank Accession No. BC071181.1 is reproduced below (SEQ ID NO: 14).

| | |
|---|---|
| 1 | gcggcggcta gggaggtggg gcgaggcgag gtttgctggg gtgaggcagc ggcgcggccg |
| 61 | ggccgggccg ggccacaggc ggtggcggcg ggaccatgga ggcggcggtc gctgctccgc |
| 121 | gtccccggct gctcctcctc gtgctggcgg cggcggcggc ggcggcggcg gcgctgctcc |
| 181 | cgggggcgac ggcgttacag tgtttctgcc acctctgtac aaaagacaat tttacttgtg |
| 241 | tgacagatgg gctctgcttt gtctctgtca cagagaccac agacaaagtt atacacaaca |
| 301 | gcatgtgtat agctgaaatt gacttaattc ctcgagatag gccgtttgta tgtgcaccct |
| 361 | cttcaaaaac tgggtctgtg actacaacat attgctgcaa tcaggaccat tgcaataaaa |
| 421 | tagaacttcc aactactggt ttaccattgc ttgttcagag aacaattgcg agaactattg |
| 481 | tgttacaaga aagcattggc aaaggtcgat ttggagaagt ttggagagga aagtggcggg |
| 541 | gagaagaagt tgctgttaag atattctcct ctagagaaga acgttcgtgg ttccgtgagg |
| 601 | cagagattta tcaaactgta atgttacgtc atgaaaacat cctgggattt atagcagcag |
| 661 | acaataaaga caatggtact tggactcagc tctggttggt gtcagattat catgagcatg |
| 721 | gatccctttt tgattactta aacagataca cagttactgt ggaaggaatg ataaaacttg |
| 781 | ctctgtccac ggcgagcggt cttgcccatc ttcacatgga gattgttggt acccaaggaa |
| 841 | agccagccat tgctcataga gatttgaaat caaagaatat cttggtaaag aagaatggaa |
| 901 | cttgctgtat tgcagactta ggactggcag taagacatga ttcagccaca gataccattg |
| 961 | atattgctcc aaaccacaga gtgggaacaa aaaggtacat ggcccctgaa gttctcgatg |
| 1021 | attccataaa tatgaaacat tttgaatcct tcaaacgtgc tgacatctat gcaatgggct |
| 1081 | tagtattctg ggaaattgct cgacgatgtt ccattggtgg aattcatgaa gattaccaac |
| 1141 | tgccttatta tgatcttgta ccttctgacc catcagttga agaaatgaga aaagttgttt |
| 1201 | gtgaacagaa gttaaggcca aatatcccaa acagatggca gagctgtgaa gccttgagag |
| 1261 | taatggctaa aattatgaga gaatgttggt atgccaatgg agcagctagg cttacagcat |
| 1321 | tgcggattaa gaaaacatta tcgcaactca gtcaacagga aggcatcaaa atgtaattct |
| 1381 | acagctttgc ctgaactctc cttttttctt cagatctgct cctgggtttt aatttgggag |
| 1441 | gtcaattgtt ctacctcact gagagggaac agaaggatat tgcttccttt tgcagcagtg |
| 1501 | taataaagtc aattaaaaac ttcccaggat ttctttggac ccaggaaaca gccatgtggg |
| 1561 | tcctttctgt gcactatgaa cgcttctttc ccaggacaga aaatgtgtag tctaccttta |
| 1621 | ttttttatta acaaaacttg ttttttaaaa agatgattgc tggtcttaac tttaggtaac |
| 1681 | tctgctgtgc tggagatcat ctttaagggc aaaggagttg gattgctgaa ttacaatgaa |
| 1741 | acatgtctta ttactaaaga aagtgattta ctcctggtta gtacattctc agaggattct |
| 1801 | gaaccactag agtttccttg attcagactt tgaatgtact gttctatagt ttttcaggat |
| 1861 | cttaaaacta acacttataa aactcttatc ttgagtctaa aaatgacctc atatagtagt |
| 1921 | gaggaacata attcatgcaa ttgtattttg tatactatta ttgttctttc acttattcag |
| 1981 | aacattacat gccttcaaaa tgggattgta ctataccagt aagtgccact tctgtgtctt |
| 2041 | tctaatggaa atgagtagaa ttgctgaaag tctctatgtt aaaacctata gtgtttgaat |
| 2101 | tcaaaaagct tatttatctg ggtaacccaa actttttctg ttttgttttt ggaagggttt |
| 2161 | ttgtggtatg tcatttggta ttctattctg aaaatgcctt tctcctacca aaatgtgctt |
| 2221 | aagccactaa agaaatgaag tggcattaat tagtaaatta ttagcatggt catgtttgaa |
| 2281 | tattctcaca tcaagctttt gcattttaat tgtgttgtct aagtatactt ttaaaaaatc |
| 2341 | aagtggcact ctagatgctt atagtacttt aatatttgta gcatacagac taatttttct |
| 2401 | aaaagggaaa gtctgtctag ctgcttgtga aaagttatgt ggtattctgt aagccatttt |
| 2461 | tttctttatc tgttcaaaga cttatttttt aagacatgaa ttacatttaa aattagaata |
| 2521 | tggttaatat taaataatag gcctttttct aggaaggcga aggtagttaa taatttgaat |
| 2581 | agataacaga tgtgcaagaa agtcacattt gttatgtatg taggagtaaa cgttcggtgg |
| 2641 | atcctctgtc tttgtaactg aggttagagc tagtgtggtt ttgaggtctc actacacttt |
| 2701 | gaggaaggca gcttttaatt cagtgtttcc ttatgtgtgc gtacattgca actgcttaca |
| 2761 | tgtaatttat gtaatgcatt cagtgcaccc ttgttacttg ggagaggtgg tagctaaaga |
| 2821 | acattctgag tataggtttt tctccattta cagatgtctt tggtcaaata ttgaaagcaa |
| 2881 | acttgtcatg gtcttcttac attaagttga aactagctta taataactgg tttttacttc |
| 2941 | caatgctatg aagtctctgc agggctttta cagttttcga agtcctttta tcactgtgat |
| 3001 | cttattctga ggggagaaaa aactatcata gctctgaggc aagacttcga ctttatagtg |
| 3061 | ctatcagttc cccgatacag ggtcagagta acccatacag tattttggtc aggaagagaa |
| 3121 | agtggccatt tacactgaat gagttgcatt ctgataatgt cttatctctt atacgtagaa |
| 3181 | taaatttgaa agactatttg atcttaaaac caaagtaatt ttagaatgag tgacatatta |
| 3241 | cataggaatt tagtgtcaat ttcatgtgtt taaaaacatc atgggaaaaa tgcttagagg |
| 3301 | ttactatttt gactacaaag ttgagttttt ttctgtagtt accataattt cattgaagca |
| 3361 | aatgaatgag tttgagaggt ttgtttttat agttgtgttg tattacttgt ttaataataa |
| 3421 | tctctaattc tgtgatcagg tacttttttt gtgggggttt tttttttgtt tttttttttt |
| 3481 | tttgttgttg tttttgggcc atttctaagc ctaccagatc tgctttatga aatccagggg |
| 3541 | accaatgcat tttatcacta aaactatttt tatataattt taagaatata ccaaaagttg |
| 3601 | tctgatttaa agttgtaata catgatttct cactttcatg taaggttatc cacttttgct |
| 3661 | gaagatattt tttattgaat caaagattga gttacaatta tacttttctt acctaagtgg |
| 3721 | ataaaatgta cttttgatga atcagggaat ttttttaaag ttggagttta gttctaaatt |
| 3781 | gactttacgt attactgcag ttaattcctt ttttggctag ggatggtttg ataaaccaca |
| 3841 | attggctgat attgaaaatg aaagaaactt aaaaggtggg atggatcatg attactgtcg |
| 3901 | ataactgcag ataaatttga ttagagtaat aattttgtca tttaaaaaca cagttgttta |
| 3961 | tactgcccat cctaggatgc tcaccttcca agattcaacg tggctaaaac atcttctggt |
| 4021 | aaattgtgcg tccatattca ttttgtcagt agccaggaga aatggggatg ggggaaatac |
| 4081 | gacttagtga ggcatagaca tccctggtcc atcctttctg tctccagctg tttcttggaa |
| 4141 | cctgctctcc tgcttgctgg tccctgacgc agagaccgtt gcctccccca cagccgtttg |
| 4201 | actgaaggct gctctggaga cctagagtaa aacggctgat ggaagttgtg ggacccactt |
| 4261 | ccatttcctt cagtcattag aggtggaagg gaggggtctc caagtttgga gattgagcag |
| 4321 | atgaggcttg ggatgcccct gctttgactt cagccatgga tgaggagtgg gatggcagca |
| 4381 | aggtggctcc tgtggcagtg gagttgtgcc agaaacagtg gccagttgta tcgcctataa |
| 4441 | gacagggtaa ggtctgaaga gctgagcctg taattctgct gtaataatga tagtgctcaa |
| 4501 | gaagtgcctt gagttggtgt acagtgccat ggccatcaag aatcccagat ttcaggtttt |
| 4561 | attacaaaat gtaagtggtc acttggcgat tttgtagtac atgcatgagt tacctttttt |
| 4621 | ctctatgtct gagaactgtc agattaaaac aagatggcaa agagatcgtt agagtgcaca |
| 4681 | acaaaatcac tatcccatta gacacatcat caaaagctta tttttattct tgcactggaa |
| 4741 | gaatcgtaag tcaactgttt cttgaccatg gcagtgttct ggctccaaat ggtagtgatt |
| 4801 | ccaaataatg gttctgttaa cactttggca gaaaatgcca gctcagatat tttgagatac |
| 4861 | taaggattat ctttggacat gtactgcagc ttcttgtctc tgttttggat tactggaata |
| 4921 | cccatgggcc ctctcaagag tgctggactt ctaggacatt aagatgattg tcagtacatt |
| 4981 | aaacttttca atcccattat gcaatcttgt ttgtaaatgt aaacttctaa aaatatggtt |
| 5041 | aataacattc aacctgttta ttacaactta aaaggaactt cagtgaattt gtttttattt |
| 5101 | tttaacaaga tttgtgaact gaatatcatg aaccatgttt tgatacccct ttttcacgtt |
| 5161 | gtgccaacgg aatagggtgt ttgatatttc ttcatatgtt aaggagatgc ttcaaaatgt |
| 5221 | caattgcttt aaacttaaat tacctctcaa gagaccaagg tacatttacc tcattgtgta |
| 5281 | tataatgttt aatatttgtc agagcattct ccaggtttgc agttttattt ctataaagta |
| 5341 | tgggtattat gttgctcagt tactcaaatg gtactgtatt gtttatattt gtaccccaaa |
| 5401 | taacatcgtc tgtactttct gttttctgta ttgtatttgt gcaggattct ttaggcttta |
| 5461 | tcagtgtaat ttctgccttt taagatatgt acagaaaatg tccatataaa tttccattga |
| 5521 | agtcgaatga tactgagaag cctgtaaaga ggagaaaaaa cataagctgt gtttccccat |
| 5581 | aagttttttt aaattgtata ttgtatttgt agtaatattc caaaagaatg taaataggaa |
| 5641 | atagaagagt gatgcttatg ttaagtccta acactacagt agaagaatgg aagcagtgca |
| 5701 | aataaattac atttttccca aaaaaaaaaa aaaaaaaaaa aaaaaaaaaa gaaaaaaaaa |
| 5761 | aaaaaa |

By “TGFβR2 polypeptide” is meant a polypeptide or fragment thereof having at least about 85% amino acid identity to GenBank Accession No. ABG65632.1 and having a biological activity of a TGFβR2 polypeptide. Biological activities of a TGFβR2 polypeptide include binding to TGFβR1 polypeptide to form a heterodimeric complex, and serine/threonine kinase activity. The sequence at GenBank Accession No. ABG65632.1 is shown below (SEQ ID NO: 15):

| | |
|---|---|
| 1 | mgrgllrglw plhivlwtri astipphvqk svnndmivtd nngavkfpql ckfcdvrfst |
| 61 | cdnqkscmsn csitsicekp qevcvavwrk ndenitletv chdpklpyhd filedaaspk |
| 121 | cimkekkkpg etffmcscss decndniifs eeyntsnpdl llvifqvtgi sllpplgvai |
| 181 | sviiifycyr vnrqqklsst wetgktrklm efsehcaiil eddrsdisst canninhnte |
| 241 | llpieldtlv gkgrfaevyk aklkqntseq fetvavkifp yeeyaswkte kdifsdinlk |
| 301 | henilqflta eerktelgkq ywlitafhak gnlqeyltrh viswedlrkl gsslargiah |
| 361 | lhsdhtpcgr pkmpivhrdl kssnilvknd ltcclcdfgl slrldptlsv ddlansgqvg |
| 421 | tarymapevl esrmnlenve sfkqtdvysm alvlwemtsr cnavgevkdy eppfgskvre |
| 481 | hpcvesmkdn vlrdrgrpei psfwlnhqgi qmvcetltec wdhdpearlt aqcvaerfse |
| 541 | lehldrlsgr scseekiped gslnttk |

By “TGFβR2 polynucleotide” is meant a polynucleotide encoding a TGFβR2 polypeptide. An exemplary TGFβR2 polynucleotide sequence is provided at GenBank Accession No. DQ377553.1. The exemplary sequence provided at GenBank Accession No. DQ377553.1 is reproduced below (SEQ ID NO: 16).

CCTCCTGGCTGGCGAGCGGGCGCCACATCTGGCCCGCACATCTGCG
CTGCCGGCCCGGCGCGGGGTCCGGAGAGGGCGCGGCGCGGAGGCGC
AGCCAGGGGTCCGGGAAGGCGCCGTCCGCTGCGCTGGGGGCTCGGT
CTATGACGAGCAGCGGGGTCTGCCATGGGTCGGGGGCTGCTCAGGG
GCCTGTGGCCGCTGCACATCGTCCTGTGGACGCGTATCGCCAGCAC
GATCCCACCGCACGTTCAGAAGTCGGGTGAGTGGTCCCCAGCCCGG
GCTCGGCGGGGCGCCGGGGGTCTTCCTGGGGTCCCCGCCTCTCCGC
TGCGCTTGACAGTCGGGCCCGGCAACCCGGCCCCCGGGCGGAAACG
AGGAAAGTTTCCCCCGCGACACTCACGCAGCCCGACTCCCGTAGCT
GCAGGGATTGTGAGTTTTTCTTGAAAAAGAGAAGGAAAGTTCAGTT
GCAAGGGGCGCGGGGCACGTTTGGTCC

As used herein, the term “rapamycin” refers to a compound (a macrocyclic triene antibiotic also known as Sirolimus) produced by the bacterium Streptomyces hygroscopicus. It inhibits the activation of T cells and B cells by reducing the production of interleukin-2 (IL-2). Rapamycin has immunosuppressant functions in humans and is especially useful in medicine for preventing organ transplant rejection such as the rejection of kidney transplants. It is also used to treat lymphangioleiomyomatosis, a lung progressive and systemic disease. Rapamycin has also been shown to inhibit proliferation of vascular smooth muscle cells migration (Poon M. et al., J Clin Invest. 1996; 98(10):2277-83). Rapamycin derivatives used according to the methods of present invention include, but are not limited to, 40-O-alkyl-rapamycin derivatives, e.g. 40-O-hydroxyalkyl-rapamycin derivatives, for example 40-O-(2-hydroxy)-ethyl-rapamycin (everolimus), rapamycin derivatives which are substituted in 40 position by heterocyclyl, e.g. 40-epi-(tetrazolyi)-rapamycin (also known as ABT578), 32-deoxo-rapamycin derivatives and 32-hydroxy-rapamycin derivatives, such as 32-deoxorapamycin, 16-O-substituted rapamycin derivatives such as 16-pent-2-ynyloxy-32-deoxorapamycin, 16-pent-2-ynyloxy-32(S or R)-dihydro-rapamycin, or 16-pent-2-ynyloxy-32(S or R)-dihydro-40-O-(2-hydroxyethyl)-rapamycin, rapamycin derivatives which are acylated at the oxygen in position 40, e.g. 40-[3-hydroxy-2-(hydroxy-methyl)-2-methylpropanoate]-rapamycin (also known as CCI779 or temsirolimus), rapamycin derivatives as disclosed in WO9802441 or WO0114387 (also sometimes designated as rapalogs), e.g. including AP23573, such as 40-O-dimethylphosphinyl-rapamycin, compounds disclosed under the name biolimus (biolimus A9), including 40-O-(2-ethoxy)ethyl-rapamycin, and compounds disclosed under the name TAFA-93, AP23464, AP23675 or AP23841; or rapamycin derivatives as e.g. disclosed in WO2004101583, WO9205179, WO9402136, WO9402385 and WO9613273.

By “subject” is meant a mammal, including, but not limited to, a human or non-human mammal, such as a bovine, equine, canine, ovine, murine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION

Without wishing to be limited by theory, it has now been shown that endothelial-to-mesenchymal transition (EndMT) plays a role in pulmonary arterial hypertension (PAH). Accordingly, the invention provides methods of treating PAH using agents that prevent or reduce EndMT.

### Therapeutic Strategy for Treating Pulmonary Arterial Hypertension

Described herein are studies demonstrating the key role of FGF signaling, let-7 miRNA expression, and TGFβ signaling in the progression of PAH by induction of endothelial-to-mesenchymal transition (EndMT) in endothelial cells and by promotion of a proliferative phenotype in smooth muscle cells

Provided herein are methods to arrest PAH by inhibiting EndMT or smooth muscle cell proliferation using a therapeutic strategy applicable to large numbers of patients. As shown in the attached figures and associated legends, EndMT plays a role in PAH and modulating this pathway fundamentally changes the course of the disease. The mechanism involves a link between FGF signaling, let-7 miRNA, and TGFβ signaling. In various embodiments, targeting this mechanism provides opportunities for the treatment and prevention of PAH.

### Endothelial-to-Mesenchymal Transition

The endothelial-to-mesenchymal transition (EndMT) is induced by activation of endothelial TGFβ signaling that occurs secondary to the loss of a protective FGF input. In healthy vessels, FGF suppresses TGFβ signaling by inducing the let-7 family of miRNAs that reduce expression of key TGFβ pathway proteins (TGFβ2, TGFβR1, Smad2). The importance of the FGF-let-7-TGFβ link is supported by human and mouse data.

Thus, in some embodiments, the TGFβ signaling is blocked by delivering let-7 miRNA into a cell. In a particular embodiment, the cell is an endothelial cell. In a particular embodiment, a systemic treatment strategy using a modified let-7 miRNA delivered to endothelial cells in targeted nanoparticles is employed. In some embodiments, the modified let-7 miRNA is mi-let-7b_{L} or mi-let-7b_{H}.

In some embodiments, the therapy is cell-type specific. Systemic inhibition of TGFβ signaling has an adverse effect by promoting inflammation and smooth muscle cell proliferation. In some embodiments, TGFβR1/2 targeted siRNAs are delivered to endothelial cells.

In some embodiments, the TGFβ signaling is activated by delivering to a cell an inhibitory polynucleotide that reduces SMC expression of FRS2α polypeptide or reduces SMC expression of a let-7 miRNA. In some embodiments, the TGFβ signaling is activated by delivering to an SMC an agent that increases the activity or level of a TGFβ signaling polypeptide. In a particular embodiment, the cell is an smooth muscle cell.

### Methods of Treatment

In some aspects, the present invention provides a method of treating pulmonary arterial hypertension and/or disorders or symptoms thereof which comprise administering a therapeutically effective amount of a pharmaceutical composition comprising an agent that modulates the activity or level of a TGFβ signaling polypeptide, a let-7 miRNA, or a FGF signaling polypeptide in a cell, to a subject (e.g., a mammal such as a human).

In particular embodiments, the agent that modulates the activity or level of a let-7 miRNA increases the activity or level of a let-7 miRNA in a cell. In some embodiments, the cell is an endothelial cell. In certain embodiments, the agent that increases the activity or level of a let-7 miRNA in a cell is a let-7 miRNA mimic. In some other embodiments, the agent is a polynucleotide encoding a let-7b miRNA. In some embodiments, the let-7 miRNA is let-7b and let-7c miRNA.

In some embodiments, the agent that modulates the activity or level of a let-7 miRNA decreases the activity or level of a let-7 miRNA in a cell. In certain embodiments, the cell is a smooth muscle cell. In some embodiments, the agent that decreases the activity or level of a let-7 miRNA in a cell is an inhibitory polynucleotide that reduces expression of let-7 miRNA. In still other embodiments, the agent that decreases the activity or level of a let-7 miRNA in a cell is a let-7 miRNA sponge or antagomir-let-7b/c. Such miRNA sponges are described in, for example, Ebert et al. RNA. 2010 November; 16(11): 2043-2050. In some embodiments, the let-7 miRNA is let-7b miRNA.

In some embodiments, the agent that modulates the activity or level of a TGFβ signaling polypeptide increases the activity or level of a TGFβ signaling polypeptide in a cell (in particular, a smooth muscle cell). In some other embodiments, the agent that modulates the activity or level of a TGFβ signaling polypeptide decreases the activity or level of a TGFβ signaling polypeptide in a cell (in particular, an endothelial cell). In some embodiments, the TGFβ signaling polypeptide is TGFβ1, TGFβ2, TGFβ3, TGFβR1, or TGFβR2. In some embodiments, the agent is siRNA and may be targeted to a TGFβ receptor.

In some embodiments, the agent that decreases the activity or level of a TGFβ signaling polypeptide is an inhibitory polynucleotide that reduces expression of a TGFβ signaling polypeptide. In some other embodiments, the agent that increases the activity or level of a TGFβ signaling polypeptide is a polynucleotide encoding a TGFβ signaling polypeptide.

In certain embodiments, the agent that modulates the activity or level of a FGF signaling polypeptide decreases the activity or level of a FGF signaling polypeptide in a cell (in particular, a smooth muscle cell). In some embodiments, the agent that modulates the activity or level of a FGF signaling polypeptide increases the activity or level of a FGF signaling polypeptide in a cell (in particular, an endothelial cell). In some embodiments, the FGF signaling polypeptide is FRS2α.

In certain embodiments, the agent that decreases the activity or level of a FGF signaling polypeptide in a cell is an inhibitory polynucleotide that reduces expression of a FGF signaling polypeptide. In some other embodiments, the agent that increases the activity or level of a FGF signaling polypeptide in a cell is a polynucleotide encoding a FGF signaling polypeptide.

In some embodiments, the subject is pre-selected by assessing the activity or level of a TGFβ signaling polypeptide or polynucleotide, a let-7 miRNA, or a FGF signaling polypeptide or polynucleotide in a sample from the subject when compared to reference levels.

The subject is pre-selected when an alteration in the activity or level of activity or level of a TGFβ signaling polypeptide or polynucleotide, a let-7 miRNA, or a FGF signaling polypeptide or polynucleotide in a sample from the subject is detected. In some embodiments, the subject is pre-selected when a decrease in the activity or level of let-7 miRNA or a TGFβ signaling polypeptide is observed relative to reference levels in an endothelial cell sample obtained from the subject. In other embodiments, the subject is pre-selected when a decrease in the activity or level of a FGF signaling polypeptide or polynucleotide, or an increase in the activity or level of let-7 miRNA or a TGFβ signaling polypeptide or polynucleotide is observed relative to reference levels in a smooth muscle cell sample obtained from the subject.

In some aspects, the subject is administered an additional agent comprising a therapeutically effective amount of an mTOR inhibitor. In some aspects of the invention, the subject is administered an additional agent comprising a therapeutically effective amount of rapamycin or any derivative thereof. In some embodiments, the therapeutically effective amount of rapamycin or any derivative thereof is used to reduce SMC proliferation and increase its differentiation alone or in combination with EC-specific therapies. In some embodiments, the agent that decreases the activity or level of a TGFβ signaling polypeptide and the additional agent are co-administered to the subject.

In other aspects of the invention, the agent that decreases the activity or level of a TGFβ signaling polypeptide is a nucleic acid capable of downregulating the gene expression of at least one gene selected from the group consisting of TGFβ1, TGFβ2, TGFβ3, TGFβR1, and TGFβR2. In some embodiments, the at least one gene is selected from the group consisting of TGFβR1, and TGFβR2.

In some instance, downregulation of the TGFβ or TGFβ receptor (TGFβR) gene expression is desired. This downregulation may result from a full or partial knock down of the gene of interest. Briefly, a gene knock down refers to a genetic technique in which one of an organism's genes is silenced, made inoperative or partially inoperative. Gene expression may be downregulated, knocked-down, decreased, and/or inhibited by various well-established molecular techniques known in the art such as, but not limited to, RNA interference (RNAi); small inhibitor RNA (siRNA), small hairpin RNA (shRNA) and Clustered Regularly Interspaced Short Palindromic Repeats (CRISPRs)).

In some embodiments, the nucleic acid is selected from the group consisting of an antisense RNA, siRNA, shRNA, and a CRISPR system. In other embodiments, the nucleic acid is combined with a therapeutically effective amount of rapamycin or any derivative thereof. In yet other embodiments, the nucleic acid is encapsulated in a nanoparticle formulated for selective delivery to an endothelial cell, in a pharmaceutically acceptable excipient. In further embodiments, the nanoparticle is a 7C1 nanoparticle.

The methods disclosed herein include administering to the subject (including a subject identified as in need of such treatment) an effective amount of an agent described herein, or a composition described herein to produce such effect. Identifying a subject in need of such treatment can be made by a health care professional and may be subjective (e.g. opinion) or objective (e.g. measurable by a test or diagnostic method, such as using the methods described herein).

The therapeutic methods of the invention, which may also include prophylactic treatment, in general comprise administering a therapeutically effective amount of one or more of the agents herein (such as an agent that modulates the activity or level of a TGFβ signaling polypeptide, a let-7 miRNA, or a FGF signaling polypeptide) to a subject (e.g., animal, human) in need thereof, including a mammal, particularly a human. Such treatment is suitable for subjects, particularly humans, suffering from, having, susceptible to, or at risk for PAH. In one embodiment, the invention provides a method of monitoring progression of treatment. The method comprises determining a level or activity of diagnostic marker (e.g., a TGFβ signaling polypeptide or polynucleotide, a let-7 miRNA, or a FGF signaling polypeptide or polynucleotide) in a subject suffering from or susceptible to PAH, in which the subject has been administered a therapeutic or effective amount of a therapeutic agent sufficient to treat PAH. The activity or level of a TGFβ signaling polypeptide or polynucleotide, a let-7 miRNA, or a FGF signaling polypeptide or polynucleotide determined in the method can be compared to a known activity or level of a TGFβ signaling polypeptide or polynucleotide, a let-7 miRNA, or a FGF signaling polypeptide or polynucleotide in either healthy normal controls, or in other afflicted patients, to establish the subject's disease status. In some embodiments, an activity or level of a TGFβ signaling polypeptide or polynucleotide, a let-7 miRNA, or a FGF signaling polypeptide or polynucleotide in an endothelial cell or smooth muscle cell sample obtained from the subject is determined. In some embodiments, a second activity or level of a TGFβ signaling polypeptide or polynucleotide, a let-7 miRNA, or a FGF signaling polypeptide or polynucleotide in the subject is determined at a time point later than the determination of the first level, and the two levels are compared to monitor the course of disease or the efficacy of the therapy. In certain embodiments, a pre-treatment activity or level of a TGFβ signaling polypeptide or polynucleotide, a let-7 miRNA, or a FGF signaling polypeptide or polynucleotide is determined prior to commencing. This pre-treatment level can then be compared to the level of a TGFβ signaling polynucleotide or polypeptide or let-7 miRNA in the subject after the treatment commences, to determine the progress or efficacy of the treatment.

### Pharmaceutical Compositions

The present invention features compositions useful for treating PAH in a pre-selected subject. The compositions include an agent that modulates the activity or level of a TGFβ signaling polypeptide, a let-7 miRNA, or a FGF signaling polypeptide in a cell.

In particular embodiments, the agent that modulates the activity or level of a let-7 miRNA increases the activity or level of a let-7 miRNA in a cell, in particular, an endothelial cell. In certain embodiments, the agent that increases the activity or level of a let-7 miRNA in a cell is a let-7 miRNA mimic. In some other embodiments, the agent is a polynucleotide encoding a let-7b miRNA. In certain embodiments, the agent that modulates the activity or level of a let-7 miRNA decreases the activity or level of a let-7 miRNA in a cell, in particular, a smooth muscle cell. In some embodiments, the agent that decreases the activity or level of a let-7 miRNA in a cell is an inhibitory polynucleotide that reduces expression of let-7 miRNA. In some embodiments, the let-7 miRNA is let-7b miRNA.

In some embodiments, the agent that modulates the activity or level of a TGFβ signaling polypeptide increases the activity or level of a TGFβ signaling polypeptide in a cell (in particular, a smooth muscle cell). In some other embodiments, the agent that modulates the activity or level of a TGFβ signaling polypeptide decreases the activity or level of a TGFβ signaling polypeptide in a cell (in particular, an endothelial cell). In some embodiments, the TGFβ signaling polypeptide is TGFβ1, TGFβ2, TGFβ3, TGFβR1, or TGFβR2.

In some embodiments, the agent that decreases the activity or level of a TGFβ signaling polypeptide is an inhibitory polynucleotide that reduces expression of a TGFβ signaling polypeptide. In some other embodiments, the agent that increases the activity or level of a TGFβ signaling polypeptide is a polynucleotide encoding a TGFβ signaling polypeptide.

In certain embodiments, the agent that modulates the activity or level of a FGF signaling polypeptide decreases the activity or level of a FGF signaling polypeptide in a cell (in particular, a smooth muscle cell). In some embodiments, the agent that modulates the activity or level of a FGF signaling polypeptide increases the activity or level of a FGF signaling polypeptide in a cell (in particular, an endothelial cell). In some embodiments, the FGF signaling polypeptide is FRS2α.

In certain embodiments, the agent that decreases the activity or level of a FGF signaling polypeptide in a cell is an inhibitory polynucleotide that reduces expression of a FGF signaling polypeptide. In some other embodiments, the agent that increases the activity or level of a FGF signaling polypeptide in a cell is a polynucleotide encoding an FGF signaling polypeptide

The composition may be administered systemically, for example, formulated in a pharmaceutically-acceptable buffer such as physiological saline. Routes of administration include, for example, subcutaneous, intravenous, intraperitoneally, intramuscular, or intradermal injections that provide continuous, sustained levels of the agent in the patient.

The amount of the therapeutic agent to be administered varies depending upon the manner of administration, the age and body weight of the patient, and with the clinical symptoms of PAH. Generally, amounts will be in the range of those used for other agents used in the treatment of PAH, although in certain instances lower amounts will be needed because of the increased specificity of the agent. A composition is administered at a dosage that decreases effects or symptoms of PAH as determined by a method known to one skilled in the art.

The therapeutic agent may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target PAH using carriers or chemical derivatives to deliver the therapeutic agent to a particular cell type (e.g., endothelial cells or smooth muscle cells). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The pharmaceutical composition of this invention could be coated or comprised in a drug-eluting stent (DES) ((Nikam et al., 2014 Med Devices 7:165-78)) that releases at a given site (such as an artery) and pace (i.e. slow release) the composition of this invention.

The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates PAH, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

In some embodiments, the composition of this invention is delivered locally from, but not limited to, the strut of a stent, a stent graft, a stent cover or a stent sheath. In some embodiments, the composition of this invention comprises a rapamycin or a derivative thereof (e.g. as described in U.S. Pat. No. 6,273,913 B1, incorporated herein by reference).

In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Polynucleotide Therapy

In some embodiments, the invention includes a method for treating, slowing the progression of, or reversing PAH, where a therapeutic polynucleotide activity or level of a TGFβ signaling polypeptide, a let-7 miRNA, or a FGF signaling polypeptide is administered to the subject. In certain embodiments, the polynucleotide is a let-7 miRNA mimic; a polynucleotide encoding let-7 miRNA, a TGFβ signaling polypeptide, or FGF signaling polypeptide; or an inhibitory polynucleotide that reduces expression of a FGF signaling polypeptide, a let-7 miRNA, or a TGFβ signaling polypeptide. Inhibitory polynucleotides include, but are not limited to siRNAs that target a polynucleotide encoding a TGFβ signaling polypeptide, a let-7 miRNA, or a FGF signaling polypeptide.

In particular embodiments, the polynucleotide therapy comprises a let-7 miRNA, a polynucleotide encoding a let-7 miRNA, or an inhibitory polynucleotide that reduces expression of a TGFβ signaling polypeptide. Such therapeutic polynucleotides can be delivered to cells of a subject having PAH. The nucleic acid molecules are delivered to the cells of a subject in a form by which they are taken up by the cells so that therapeutically effective levels of the inhibitory nucleic acid molecules are contained within the cells.

Introduction of nucleic acids into cells may be accomplished using any number of methods available in the art. For example, transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid or miRNA (or a precursor to the miRNA) as described can be cloned into a retroviral vector where expression can be driven from its endogenous promoter, from the retroviral long terminal repeat, or from a promoter specific for a target cell type of interest. In some embodiments, the target cell type of interest is an endothelial cell. Other viral vectors that can be used to introduce nucleic acids into cells include, but are not limited to, vaccinia virus, bovine papilloma virus, or herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit expression of TGFβ signaling polypeptide.

Non-viral approaches can also be employed for the introduction of the therapeutic to a cell of a patient requiring treatment of PAH. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the therapeutic polynucleotide is selectively targeted to an endothelial cell. In some other embodiments, the therapeutic polynucleotide is expressed in an endothelial cell using a lentiviral vector. In still other embodiments, the therapeutic polynucleotide is administered intravenously. In some embodiments, the therapeutic polynucleotide contains one or more chemical modifications that reduce immunostimulation, enhance serum stability, increase specificity, and/or improve activity, while still retaining silencing activity. Such chemical modifications are described in, for example, Foster et al., RNA. 2012 March; 18(3): 557-568. In some embodiments, the therapeutic polynucleotide contains one or more chemical modifications to prevent degradation, as described in Chen et al., Cell Reports 2012; 2(6)1684-1696.

In a particular embodiment, the therapeutic polynucleotide is selectively delivered to endothelial cells using nanoparticles formulated for selective targeting to endothelial cells, such as a 7C1 nanoparticle. Selective targeting or expression of polynucleotides to an endothelial cell is described in, for example, Dahlman et al., Nat Nanotechnol. 2014 August; 9(8): 648-655.

In some other embodiments, the therapeutic polynucleotide is selectively targeted to a smooth muscle cell. The therapeutic polynucleotide can be selectively delivered to a smooth muscle cell using tissue factor-targeted nanoparticles that can penetrate and bind stretch-activated vascular smooth muscles as described in Lanza et al., Circulation. 2002 Nov. 26; 106(22):2842-7.

In General

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Attorney Docket No. 047162-7221US1(01568) Preliminary Amendment

What is claimed is:

1. A method of treating pulmonary arterial hypertension (PAH) in a subject, the method comprising administering to the subject an agent that modulates the activity or level of let-7 miRNA in an endothelial cell in the subject, thereby treating PAH in the subject.

2. A method of treating pulmonary arterial hypertension (PAH) in a subject, the method comprising administering to the subject an agent that decreases, in an endothelial cell in the subject, the activity or level of a endothelial TGFβ signaling polypeptide or TGFβ peptide receptor selected from the group consisting of TGFβ1, TGFβ2, TGFβ3, TGFβR1, and TGFβR2, thereby treating PAH in the subject.

3. The method of claim 1, wherein the agent is selectively delivered to an endothelial cell in the subject.

4. The method of claim 3, wherein the agent is in a nanoparticle.

5. The method of claim 4, wherein the nanoparticle is a 7C1 nanoparticle.

6. The method of claim 3, wherein the agent is selectively delivered to a smooth muscle cell in the subject.

7. The method of claim 1, wherein the agent is administered intravenously.

8. The method of claim 1, wherein the agent that increases the activity or level of let-7 miRNA is selected from the group consisting of human let-7b miRNA and human let-7c miRNA.

9. The method of claim 1, wherein the agent that modulates the activity or level of let-7 miRNA is a pharmaceutical composition comprising an effective amount of a let-7 miRNA in a nanoparticle formulated for selective delivery to an endothelial cell, in a pharmaceutically acceptable excipient.

10. The method of claim 9, wherein the let-7 miRNA comprises a chemical modification that increases stability of the miRNA and/or reduces an immune response to the miRNA in a subject.

11. The method of claim 10, wherein the chemical modification is a 2′-O-methyl modification.

12. The method of claim 9, wherein the let-7 miRNA is selected from the group consisting of human let-7b miRNA and human let-7c miRNA.

13. The method of claim 12, wherein the nanoparticle is a 7C1 nanoparticle.

14. The method of claim 2, wherein the agent that decreases the activity or level of a TGFβ signaling polypeptide is an inhibitory polynucleotide that reduces expression of the TGFβ signaling polypeptide.

15. A method of treating pulmonary arterial hypertension (PAH) in a subject, the method comprising administering to the subject an agent that decreases in an endothelial cell in the subject the activity or level of FRS2α, thereby treating PAH in the subject.

16. The method of claim 15, wherein the agent that decreases the activity or level of FRS2α is an inhibitory polynucleotide that reduces expression of a FRS2α polypeptide.

17. The method of any one of claim 15, wherein the decrease in the activity or level of the FRS2α polypeptide promotes smooth muscle cell proliferation.

18. The method of claim 1, further comprising providing to the subject a second therapeutic agent comprising an mTOR inhibitor.

19. The method of claim 18, wherein the mTOR inhibitor is rapamycin.
